(19) United States
(12) Patent Application Publication
(10) Pub. No.: US 2026/0151569 A1
(43) Pub. Date: Jun. 4, 2026

(54) DRUG DELIVERY DEVICE AND ELECTRONIC UNIT

(71) Applicant: Sanofi, Paris (FR)

(72) Inventors: Stefan Alt, Frankfurt am Main (DE); Tim Glasser, Rüsselsheim (DE); Michael Helmer, Frankfurt am Main (DE); Stephan Mücke, Rüsselsheim (DE); Peter Nober, Rüsselsheim (DE); Michael Schabbach, Frankfurt am Main (DE); Martin Vitt, Frankfurt am Main (DE)

(21) Appl. No.: 18/706,830

(22) PCT Filed: Nov. 1, 2022
(86) PCT No.: PCT/EP2022/080403

§ 371 Date: May 2, 2024

(30) Foreign Application Priority Data
Nov. 3, 2021 (EP) 21315218.4

(51) Int. Cl. A61M 5/315; A61M 5/32

(52) U.S. Cl. CPC A61M 5/3158; A61M 5/3202; A61M 2205/8206

(57) ABSTRACT

The present disclosure relates to drive unit (3) of a drug delivery device (1) configured for setting and dispensing of a dose of a medicament, the drive unit (3) comprising: a drive unit housing (9) extending along a longitudinal axis (A), the drive unit housing (9) comprising a distal end (4) and a proximal end (5), a first interface (110) provided at the distal end (4), configured to connect with at least one of a container unit (2) and a protective cap (14), the first interface (110) comprising at least a first electrical contact element (111, 112), an electronic unit (70) arranged on or inside the drive unit housing (9), a first elongated device component (40) extending along the longitudinal axis (A), a first electrical conductor path (141) located on or inside the first elongated device component (40), the first electrical conductor path (141) comprising a first path distal end (142) and a first path proximal end (143), wherein the first path proximal end (143) is connected to the electronic unit (70) and wherein the first path distal end (142) is connected to the first electrical contact element (111, 112), wherein the electronic unit (70) is operable to execute an electronically implemented function in response to receive an electric detection signal via the first electrical conductor path (141).

### FIELD

The present disclosure relates to a drive unit of a drug delivery device, in particular to a drive unit of a handheld injection device. The disclosure further relates to such a drug delivery device or injection device equipped with an electronic unit.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, e.g. injection devices and needle based injection system (NIS) devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

With reusable injection devices a medicament container, i.e. a cartridge has to be replaced when the medicament has been depleted. Here, the medicament container, e.g. a vitreous cartridge filled with the medicament has to be replaced. Some primary medicament containers, e.g. in form of a cartridge filled with the medicament are preassembled in a housing component of the injection device, such as a cartridge holder.

In either way and with reusable devices it should be ensured, that only a dedicated medicament container or dedicated housing component preassembled with such a medicament container can be used with a dedicated drive unit or drive mechanism of an injection device.

Generally, there may be provided a large variety of primary medicament containers that differ with regards to their filling volume or with regard to the pharmaceutical substance or concentration of the medicament. For replacement of a medicament container it must be ensured, that an injection device or drive mechanism can only be used in conjunction with a dedicated medicament container intended for use with this particular type of injection device or drive mechanism. Unintended cross use of non-matching pairs of medicament containers and drive mechanisms or injection devices has to be avoided.

Some injection devices also comprise an electronic unit by way of which setting and/or dispensing of a dose can be monitored and/or recorded over time. Such electronic units may provide communication with an external electronic device, e.g. a personal electronic device such as a smart phone or smart watch. Such electronic units are typically implemented at or near a proximal end of a handheld injection device, where sufficient construction space is provided.

With such examples it is and remains quite challenging to provide correct information about the specific type of medicament to the electronic unit. It is hence desirable to automatically provide information about the primary medicament container to the electronic unit. Furthermore, it is a particular aim to improve patient safety and to provide a drug delivery device with enhanced security functions.

### SUMMARY

According to one aspect the present disclosure relates to a drive unit of a drug delivery device. The drive unit is configured for setting and dispensing of a dose or of multiple doses of a medicament. The drive unit comprises a drive unit housing. The drive unit housing extends along a longitudinal axis. The drive unit housing may comprise an elongated structure extending along the longitudinal axis. The drive unit housing comprises a distal end and a proximal end. The distal end and the proximal end are opposite longitudinal ends of the drive unit housing.

The drive unit further comprises a first interface provided at the distal end. The first interface is configured to connect with at least one of a container unit and a protective cap of the drug delivery device. The first interface comprises at least a first electrical contact element. The drive unit further comprises an electronic unit arranged on or inside the drive unit housing. Typically, the electronic unit is provided at or near the proximal end of the drive unit housing. The drive unit further comprises a first elongated device component extending along the longitudinal axis.

The drive unit further comprises a first electrical conductor path located on or inside the first elongated device component. The first electrical conductor path comprises a first path distal end and a first path proximal end. The first path proximal end is connected to the electronic unit and the first path distal end is connected to the first electrical contact element. This way and by the first electrical conductor path the first electrical contact element as provided at or in the first interface at or near the distal end of the drive unit housing can be electrically connected to the electronic unit.

The electronic unit is operable to execute an electronically implemented function in response to receive an electric detection signal via the first electrical conductor path. This way, there can be provided a galvanic, i.e. conducting electrical connection between the first interface provided at the distal end of the drive unit housing and the electronic unit, which is typically provided at or near the opposite, hence at the proximal end of the drive unit housing.

The first interface of the drive unit may be implemented as a mechanical interface and/or as a hybrid electro-mechanical interface. The first interface is typically configured to connect or to engage with a complementary shaped counter interface of a container unit of the drug delivery device, wherein the container unit is sized to accommodate a drug container.

Typically, the container unit is provided with a first counter contact element at the first counter interface to engage with the first contact element of the first interface. In this way an electrical contact between the first contact element and the first counter contact element and hence the presence of a container unit, i.e. a drug container and/or a cartridge holder receiving such a drug container can be electrically detected by the electronic unit.

The electronic unit may provide a large variety of electronically implemented functions. With some examples the electronically implemented function may comprise at least one of a release or activation of an electromechanical interlock. The electromechanical interlock or locking mechanism may be operable to deactivate or to impede mechanical actuation of the drive unit. Hence with an activated electromechanical interlock at least one of setting and dispensing of a dose is effectively blocked. The electronic unit may be suitably coupled with the electromechanical interlock so as to unlock the drive unit. With other examples the electronically implemented function comprises generating of a user-perceptible alert, which may include at least one of a visual alert, an acoustic alert or a haptic alert, e.g. when a wrong or non-matching container unit should be attached to the drove unit.

With further examples the electronically implemented function includes setting up a communication link and/or a communication with an external electronic device via the communication link. Here, the electronic unit may be operable to communicate with the external electronic device on the basis of a wireless communication protocol. The external electronic device may be implemented or configured to transmit a request to the electronic unit, which is processable by the electronic unit.

With some configurations, wherein a correct container unit is assembled and/or attached to the first interface of the drive unit there may be provided a respective container unit identifying detection signal via the first electrical contact element and the first electrical conductor path. Then, the electronic unit receiving such an electric detection signal may approve a request as received from an external electronic device. Accordingly, the electronic unit may appropriately deploy or set up when receiving an electric detection signal via the first electrical conductor path being indicative of a correct or matching container unit.

The electric detection signal may be indicative of one of a plurality of generally available container units that can be used with the drive unit in principle. The specific electric detection signal received by the electronic unit may be used and/or processed by the electronic unit to calibrate and/or to deploy a proper and intended function of the electronic unit.

According to a further example the drive unit comprises a second electrical contact element and a second electrical conductor path. The second electrical conductor path is electrically connected to the second electrical contact element. The second electrical contact element is electrically isolated from the first electrical contact element. Likewise, the second electrical conductor path is electrically isolated from the first electrical conductor path.

The second electrical conductor path comprises a second path distal end and a second path proximal end. The second path proximal end is connected to the electronic unit. The second path distal end is connected to the second electrical contact element. This way, there are provided two separate and electrically isolated conductor paths extending from the first interface to the electronic unit.

With a first conductor path and with a second conductor path electrically isolated from the first conductor path, there can be provided a twofold electrical connection for the electronic unit. Accordingly, the first electrical conductor path may be configured to transmit a first electric detection signal to the electronic unit. Likewise, the second electrical conductor path may be configured to transmit or to conduct a second electric detection signal to the electronic unit. Insofar and with at least a first and a second electrical conductor path extending from the first interface to the electronic unit and galvanically connecting the first interface with the electronic unit there can be transmitted and processed a variety of electric detection signals.

According to a further example the second path distal end is connectable to the first path distal end by a first counter interface of one of the container unit and the protective cap when the first counter interface is engaged with the first interface. Here, the first path distal end and the second path distal end are electrically connectable with each other by the first counter interface as provided by one of the container unit and the protective cap.

This way, the first path distal end and the second path distal end may be connected in series so as to form a closed conductor loop thereby connecting the first electrical conductor path and the second electrical conductor path. The first counter interface may be provided with a contact conductor providing an electrical connection between the first contact element and the second contact element. Typically, and when the first interface of the drive unit engages with the first counter interface of one of the container unit and the protective cap the contact conductor gets in mechanical and electrical connection with both, the first contact element and the second contact element. An open circuit as provided by the first and second electrical conductor path individually connected with the electronic unit may then be closed.

According to another example the second path distal end is electrically connectable to the first path distal end by an electrical connector movably arranged on or in the first interface. Typically, the electrical connector is movable between a release position and a contact position. In the release position the electrical connector is out of contact with regard to at least one of the first contact element and the second contact element. In the contact position the electrical connector is in electrical contact with both, the first contact element and the second contact element.

With some examples the electrical connector is movable from the release position into the contact position against the action of a contact spring. The electrical connector may be urged from the release position into the contact position in the course of assembling or engaging the first counter interface with the first interface. When released, i.e. when the first counter interface of one of the container unit and the protective cap is detached or released from the first interface of the drive unit the electrical connector may return into the release position under the action of the contact spring.

The electric circuit previously formed by the electrical connector, the first electrical conductor path, the second electrical conductor path and the electronic unit may be then interrupted. Generally, and when there is no electric connection between the first contact element and the second contact element there may be provided an open or interrupted electric circuit, which is detectable by the electronic unit. Here, and with an open circuit the electronic unit may be configured to enter into a sleep or idle mode, e.g. in order to save energy.

According to another example the second electrical conductor path is located on or inside the first elongated device component. Hence, the first and the second electrical conductor path are located on one and the same elongated device component. The elongated device component may be of tubular shape. It may comprise a sleeve or a housing portion of the drive unit. The first electrical conductor path may extend along a first sidewall section of the first device component. The second electrical conductor path may extend along a second sidewall section of the first elongated device component. The first and the second sidewall sections may be circumferentially or tangentially offset from each other.

With further examples the first and the second electrical conductor paths are spatially separated from each other e.g. with regards to the longitudinal direction and/or with regards to the circumferential direction. The first and the second electrical conductor paths may be located on geometrical oppositely located sidewall sections of the first elongated device component. With some examples the first electrical conductor path is provided on one of an inside and an outside of a sidewall of the first elongated device component. The second electrical conductor path may be then provided on the other one of the inside and the outside of the sidewall of the first elongated device component.

Typically, the first elongated device component is made of an electrically non-conducting material. The first elongated device component may be implemented as an injection molded plastic component of low or neglectable electrical conductivity. With some examples at least one of the first and the second electrical conductor paths comprises a metallized structure or metallic structure provided on or inside the first elongated device component. Typically, a portion of the first elongated device component, e.g. a sidewall portion may be coated, printed, laminated or bonded with a metallic or metallized structure. With some examples at least one of the first and second electrical conductor paths may be provided on a flexible substrate, such as a flexible foil adhesively attached or bonded on the first elongated device component.

With other examples at least one of the first and the second electrical conductors is insert molded in the first elongated device component implemented or manufactured as an injection molded plastic component.

With another example the drive unit comprises a second device component movable relative to the first device component for at least one of setting of the dose and dispensing of the dose of the medicament. The electronic unit is arranged on or in the second device component. A first electrical conductor path extension is located on or inside the second device component and is electrically connected to the electronic unit. It is further electrically connected to the first electrical conductor path provided on the first elongated device component. Typically, the first electrical conductor path extension and the first electrical conductor path are electrically connected across an interface between the first device component and the second device component. This way, the electronic unit can be electrically connected to the first electrical conductor path. It is electrically connected to the first electrical conductor path via the first electrical conductor path extension.

By way of the first electrical conductor provided on or inside the first elongated device component and by way of the first electrical conductor path extension provided in or on the second device component and being in permanent electrical connection with the first electrical conductor path a persistent and permanent electrical connection can be provided between the electronic unit and the first electric conductor path.

Typically, the first and the second device component are arranged in an at least partially overlapping or interleaved configuration. With some examples at least a portion of an outside facing surface of the first device component is located inside an inside surface of the first device component; or vice versa.

With some examples, wherein the first device component is located inside the second device component the first electrical conductor path may be provided on an outside facing surface of the first elongated device component. Here, the first electrical conductor path extension is provided on an inside facing surface of the second device component receiving or accommodating at least a portion of the first device component.

According to a further example there is provided a second electrical conductor path extension on or inside the second device component. The second electrical conductor path extension is electrically connected to the electronic unit and is further electrically connected to the second electrical conductor path. Typically, the first and the second electrical conductor path extensions may be provided on one and the same device component. The second electrical conductor path extension may be implemented in close correspondence to the first electrical conductor path extension. Insofar, any features, effects and benefits as described above with regards to the first electrical conductor path extension may equally apply to the second electrical conductor path extension.

Typically, and with some examples the first and/or the second electrical conductor path extensions comprise an elongated structure. A distal end of at least one of the first and second electrical conductor path extensions may be in electrical contact with the respective first or second electrical conductor path as provided on or in the first elongated device component. An opposite end, hence a proximal end of the first and/or second electrical conductor path extension may be directly connected to the electronic unit.

With further examples it may be a proximal end of at least one of the first and second electrical conductors that is electrically connected with a portion of the first or second electrical conductor path extensions. The portion of the first and/or second electrical conductor path extension being electrically connected to the respective first or second electrical conductor path may be located between the distal end and the proximal end of the first or second electrical conductor path extensions. This allows for a movement of the first device component relative to the second device component along the longitudinal axis thereby maintaining an electrical contact between the first electrical conductor path and the first electrical conductor path extension and/or between the second electrical conductor path and the second electrical conductor path extension.

According to a further example of the drive unit the second device component is movable relative to the first device component along a first movement direction. At least one of the first electrical conductor path and the first electrical conductor path extension extends along the first movement direction or parallel to the first movement direction.

With some examples the second device component may be movable relative to the first device component along the longitudinal axis. It may be in a sliding engagement with the first elongated device component. Then and for maintaining an electrical contact between the first electrical conductor path and the first electrical conductor path extension it is of particular benefit when at least one of the first electrical conductor path and the first electrical conductor path extension also extends along the respective movement direction. In this way and when the second device component is subject to a movement relative to the first device component the electrical contact between the first electrical conductor path and the first electrical conductor path extension can be maintained.

Also here, the same may be valid for the second electrical conductor path and the second electrical conductor path extension.

According to another example the first electrical conductor path and the first electrical conductor path extension are permanently electrically connected via a first sliding contact. A sliding contact provides a persistent electrical connection between the first electrical conductor path and the first electrical conductor path extension even when the first elongated device component is subject to a movement relative to the second device component; or vice versa.

According to another example also the second electrical conductor path and the second electrical conductor path extension are permanently electrically connected via a second sliding contact. Insofar, the electrical interfaces provided between first and second electrical conductor paths and first and second electrical conductor path extensions are substantially identically or correspondingly shaped.

According to another example the first sliding contact comprises at least one mechanically biased contact conductor connected to one of the first electrical conductor path and the first electrical conductor path extension. The mechanically biased contact conductor is in sliding surface contact with the other one of the first electrical conductor path and the first electrical conductor path extension. The mechanically biased contact conductor may be biased in radial direction in case the first electrical conductor path and the respective first electrical conductor path extension overlap in radial direction.

The mechanically biased contact conductor may be resiliently deformable, e.g. in radial direction. With other examples the mechanically biased contact conductor may be provided with a biasing spring by way of which a contact pad or contact pin of the contact conductor is kept in mechanical engagement and hence in an electrically conducting surface contact with the first electrical conductor path or first electrical conductor path extension. In this way, a rather reliable and persistent as well as a permanent electrical connection can be provided between the first electrical conductor path and the first electrical conductor path extension.

According to an example the first device component and the second device component are threadedly engaged. The second device component is movable relative to the first device component along a helical path. With such an example at least one of the first electrical conductor path and the first electrical conductor path extension comprises a helical structure on a sidewall of one of the first device component and the second device component. The helical structure, i.e. the threaded engagement between the first device component, and the second device component comprise the same lead. In this way and when e.g. the second device component is subject to a helical movement relative to the first device component, e.g. during setting or dispensing of a dose, the first electrical conductor path and the first electrical conductor path extension can remain in electrical contact. In this way there can be provided a persistent and durable electrical connection between the first and/or second contact element of the first interface of the drive unit and the electronic unit, e.g. provided at or near the proximal end of the drive unit.

According to a further example the first electrical conductor path extends along the longitudinal axis and the first electrical conductor path extension extends along the longitudinal axis. The sliding contact comprises a conductor disc with an annular or arc-shaped contact conductor. Here and with such an example the conductor disc may be non-rotationally connected or engaged e.g. with the first elongated device component. When the second device component is subject to a helical motion relative to the first device component the conductor disc may follow only the longitudinal part of such a helical motion. It may slide along the first elongated electrical conductor path, e.g. provided on an inside of the sidewall of the first elongated device component.

A semicircular and/or arc-shaped sliding contact, e.g. provided on a proximally facing side of the conductor disc may be in permanent axial contact or axial engagement with the first electrical conductor path extension. The annular or arc-shaped contact conductor as provided on the conductor disc is operable to provide or to support a permanent electrical contact with the first electrical conductor path extension even when the second device component is subject to a rotational movement relative to the first elongated device component.

The implementation of a conductor disc with an annular or arc-shaped contact conductor is beneficial to support and to maintain an electrical contact between the first electrical conductor path and the first electrical conductor path extension when the second device component is subject to a helical motion relative to the first elongated device component during and/or for at least one of setting of the dose and dispensing of the dose and wherein the second device component is subject to a non-rotational and hence purely longitudinal sliding motion relative to the first elongated housing component during the other one of dose setting and dose dispensing.

According to another example the first sliding contact comprises an annular shaped contact conductor on one of the first device component and the second device component. The first sliding contact further comprises a tangentially extending and radially biased conductor branch on the other one of the first device component and the second device component. Here, the conductor branch is in surface contact with the annular shaped contact conductor. With such an implementation the first sliding contact provides a permanent electrical connection between the first elongated housing component and the second housing component when the first elongated housing component is subject to a non-axial rotational movement relative to the second housing component with the longitudinal axis as an axis of rotation.

Such an implementation is of particular benefit to maintain an electrical contact between the first electrical conductor path and the first electrical conductor path extension when the first elongated device component is longitudinally fixed to the second device component as is only allowed to rotate relative to the second device component with the longitudinal axis as an axis of rotation.

With a further example also the second sliding contact comprises an annular shaped contact conductor on one of the first device component and the second device component and further comprises a tangentially extending and radially biased conductor branch on the other one of the first device component and the second device component.

Also here, the conductor branch of the respective second sliding contact is in surface contact with the annular shaped contact conductor. Typically, the first sliding contact is then longitudinally offset from the second sliding contact thus providing electrical isolation between the first and the second sliding contact.

According to another example the drive unit further comprises an electromechanical actuator electrically connected to the electronic unit by a first electrical control path and a second electrical control path. The first electrical control path and the second electrical control path are electrically isolated from each other. The electronic unit is operable to selectively activate or to deactivate the electromechanical actuator by generating and transmitting at least one control signal to the electromechanical actuator by the first and the second electrical control paths. The electromechanical actuator may comprise at least one of an electric drive and an electromagnet. The electronic unit is configured to deliver and/or to transmit a control voltage and/or a control current to the electromechanical actuator in order to activate or to deactivate the actuator. The electromechanical actuator may belong to an electromechanically implemented interlock operable to impede or to block at least one of setting of the dose and dispensing of the dose.

By activating the electromechanical actuator and/or the interlock may be activated thereby disabling at least one of setting of the dose and dispensing of the dose. The electromechanical actuator may comprise a latch to engage with a complementary-shaped counter latch of a movable component of the drive unit. By activating the actuator movement of the movable component can be impeded or blocked.

By way of the first and second electrical control paths the electromechanical actuator can be arranged at a particular distance from the electronic unit. This provides a rather large degree of flexibility for arranging and assembling the electromechanical actuator. The electromechanical actuator may be located at or near the distal end of the drive unit housing or it may be located e.g. between the distal end and the proximal end of the drive unit housing. The electronic unit, the first electrical control path, the electromechanical actuator and the second control path may form or constitute a closed electric circuit.

The circuit may be closed or opened by a switch arrangement of the electronic unit. With other examples there may be provided a relay in the circuit of the electromechanical actuator. By way of the electronically controllable electromechanical actuator there can be provided an electrically implemented interlock arrangement for selectively locking or unlocking the drive unit.

According to a further example the electromechanical actuator is arranged on or in the first device component and is operable to block or to impede a movement of the first device component relative to the second device component when activated.

With other examples the electromechanical actuator is arranged on or inside the second device component. It may be then likewise operable to block or to impede a movement of the first device component relative to the second device component when activated. When the electromechanical actuator is arranged on or in the first device component it is operable to engage with a recessed structure of the second device component. When the electromechanical actuator is arranged on or in the second device component it is operable to engage with a recessed structure provided on the first device component.

With an alternative example, the interlock between the first and the second device component is unlocked by activating the electromechanical actuator. The first and second device components may be locked to each other when the electromechanical actuator is deactivated. Then, the mutually locked configuration may be default configuration.

According to a further example the first control path is located on or inside the second device component. A first control path extension is located on or inside the first device component. The first electrical control path and the first control path extension are permanently electrically connected via a sliding contact. With further examples the electromechanical actuator may be provided on a third device component movable relative to the second device component on which the electronic circuit is located. Then, the first electrical control path is located on or inside the second device component and the first control path extension is located on or inside the third device component. Also here, the first electrical control path and the first control path extension are permanently electrically connected via a sliding contact.

With other examples there might not be a permanent electrical connection between the first electrical control path and the first control path extension. Here, a respective electrical connection may be provided in only one or in numerous selected dedicated positional states of the first device component and the second device component, e.g. when the drive unit is in a zero dose configuration, which is obtained at the end of a dispensing procedure or which is provided before setting of a dose.

The sliding contact between the first control path and the first control path extension provides an increased degree of flexibility for arranging the electromechanical actuator at or on a device component that differs from the device component on which the electronic unit is located.

According to a further example the electronic unit is operable to process the electronic detection signal received from at least one of the first electrical conductor path and the second electrical conductor path. The electronic unit is further operable to generate and to transmit an electric control signal to the electromechanical actuator via at least one of the first electrical control path and the second electrical control path on the basis of a processing of the electric detection signal.

With some examples and when the electric detection signal is indicative of a wrong container unit assembled or attached to the first interface the electronic unit is operable to activate and/or to switch the electromechanical actuator via the first and the second electrical conductor path, in order to lock the drive unit. Then and in response to a detection of a wrong or unsuitable container unit connected to the distal end of the drive unit housing at least one of setting of the dose and dispensing of the dose is effectively impeded or locked. This way, patient safety can be effectively increased.

With some examples and by default the electromechanical actuator may be activated and has to be deactivated by the electronic unit transmitting a respective control signal to the electromechanical actuator via the first and the second electrical control paths. This way and per default, at least one of setting of the dose and dispensing of the dose is initially blocked. Unlocking of the interlock requires detection of a correct or suitable container unit attached to the distal end of the drive unit. This way and when a suitable electronic detection signal is received from at least one of the first and second electrical conductor paths the electronic unit is operable to which the state of the electromechanical actuator so as to unlock operation of the drive unit.

According to another example the first electrical control path is electrically connected to the first electrical conductor path. Likewise, the second electrical control path is electrically connected to the second electrical conductor path. In this way, an electrical path for electrically sensing or detecting a container unit attached or assembled to the distal end of the drive unit and a respective conductor path by way of which the electromechanical actuator is controllable may share a common electrically conductive structure on or in at least one of the first device component and the second device component.

In this way the number of individual electrical conductor paths and/or electrical conductor path extensions can be reduced. With some examples the first electrical control path and the first electrical conductor path may overlap or even entirely coincide. With other examples, the first electrical conductor path may branch off from the first electrical control path; and vice versa. The same configuration may apply to the second electrical control path and the second electrical conductor path.

According to a further example the electric detection signal distinguishes from the electric control signal via at least one of an amplitude and a frequency. Hence, a control signal for switching the electromechanical actuator from one state into another state may be larger in amplitude compared to the electric detection signal. Providing an electrical contact with the first electrical contact element and optionally also with the second electrical contact element generates an electric detection signal which is of a comparatively low amplitude, which is below an activation threshold required for switching the electromechanical actuator.

In response to the detection of a correct or wrong cartridge the electronic unit may be operable to generate the electric control signal and to transmit the electric control signal to the electromechanical actuator. Here, the control signal is of larger amplitude or magnitude compared to the detection signal received from the first electrical contact element and optionally from the second electrical contact element.

In the same or like manner or as an alternative the electric detection signal by way of which the presence of a particular container unit can be detected distinguishes from the electric control signal by a frequency. Here, activation or switching of the electromechanical actuator may require an electric control signal of a given frequency. A continuous and substantially non-frequent signal as provided from the first electrical contact element and optionally from the second electrical contact element may be therefore unsuitable to activate or to deactivate the electromechanical actuator.

According to a further example the drive unit comprises a second interface at the distal end. The second interface is configured to connect with a protective cap and comprises a first supply contact and a second supply contact. The drive unit further comprises a first electric supply conductor path located on or inside the first elongated device component. The first electric supply conductor path comprises a distal end and a proximal end. The proximal end is connected to the electronic unit. The distal end is connected to the first supply contact. The drive unit further comprises a second electric supply conductor path located on or inside the first elongated device component. The second electric supply conductor path comprises a distal end and a proximal end. Also here, the proximal end is connected to the electronic unit. The distal end is connected to the second supply contact. The first and the second supply conductor paths are configured to supply the electronic unit with electric power or electric energy that can be provided by a secondary or primary battery of the protective cap.

With other examples the first supply conductor path may be located on or inside another device component that distinguishes from the first elongated device component. The first and second electric supply conductor paths may be provided on a fourth device component, e.g. on or inside an elongated housing component of the drive unit.

According to a further example the electronic unit comprises a rechargeable battery. The rechargeable battery is rechargeable by an electric energy source of the protective cap when the protective cap is connected to the second interface, when a first electric supply path of the protective cap is connected to the first supply contact and when a second electric supply path of the protective cap is connected to the second supply contact.

The energy source of the protective cap may be implemented as a rechargeable battery. It may be provided as a kind of a power bank to provide electric power or electric energy to the electronic unit when assembled to the drive unit. Like the first and second electrical conductor paths the first and second electric supply conductor paths may also comprise a respective supply conductor path extension, respectively, wherein the supply conductor path extensions are directly connected to the rechargeable battery of the electronic unit and wherein the supply conductor path extensions and the supply conductor paths are permanently or at least temporally electrically connected by a contact, e.g. by a sliding contact. In this way the electronic unit located on the movable second device component can be supplied with electric energy.

In principle, the electric supply conductor path(s) and the optional supply conductor path extension(s) may be implemented in the same or like manner compared to the first and second electrical conductor paths and electrical conductor path extensions.

According to another aspect the present disclosure also relates to a drug delivery device for setting and dispensing of a dose of a medicament. The drug delivery device comprises a drive unit as described above. The drug delivery device further comprises a container unit sized to accommodate a drug container. The container unit comprises a first counter interface configured to connect to the first interface of the drive unit. The first counter interface further comprises a first counter contact element. The first counter contact element is configured to electrically connect to at least one of the first contact element and the second contact element and/or to provide an electrical contact between the first contact element and the second contact element.

When the first counter contact element is configured to electrically connect to at least one of the first contact element and the second contact element of the drive unit an electrical property of the first counter contact element can be detected by the electronic unit. Here, different container units may distinguish from each other. They may be characterized by a geometric structure and/or by electrically measurable properties of the first counter contact element.

Moreover, there may be provided a large variety of contact elements at or in the first interface of the drive unit. The container unit may be electrically encoded by at least one of a geometric position and a geometric shape of the first counter contact element. With one example of a container unit the first counter contact element may be provided at a first position. With a second container unit the first counter contact element may be provided at a second position that differs from the first position. When appropriately connected with the drive unit the first counter contact element of the first container unit may be exclusively in contact with the first electrical contact element. When the second container unit is connected to the first interface the first counter contact element may be in electrical contact with e.g. the second electrical contact element of the drive unit. This way, container units that distinguish by the position or shape of the first counter contact element can be electronically detected by the electronic unit.

With other examples the first counter contact element provides an electrical contact between the first electric contact element and the second electric contact element. It may close the electric circuit provided by the first and second contact elements and the respective first and second electrical conductor paths.

There may be provided numerous first, second and also third and/or fourth electrical contact element on or in the first interface of the drive unit that distinguish by their longitudinal and/or circumferential position. The first counter contact element of the container unit may be electrically encoded so as to provide electrical contact between two selected electrical contact elements of the drive unit.

With one example the first counter contact element of the container unit may provide electrical contact between a first and a second electrical contact element of the drive unit. With another example the first counter contact element provides electrical contact between a first and a third electric contact element of the first interface of the drive unit.

According to a further example the drug delivery device is implemented as a pen-type injector. It may comprise a hand-held drug delivery device configured for injecting a medicament by way of an injection needle penetrating the skin of a patient. The drug delivery device may comprise a drug container filled with the medicament and being arranged inside the container unit Typically, the drive unit comprises a mechanical connector at the distal end of the drive unit housing. The container unit comprises a complementary shaped connector or a fastener at a proximal end. Hence, a proximal end of the container unit is connectable, e.g. releasably connectable to the distal end of the drive unit. The mutually corresponding connectors or fasteners as provided at the distal end of the drive unit and provided at the proximal end of the container unit may be implemented as one of a snap connection, a bayonet connection and a screwed connection. Typically, one of the drive unit and the container unit comprises an insert complementary shaped to a receptacle of the other one of the drive unit and the container unit. The insert is sized and shaped to fit into the receptacle in order to securely fasten the container unit to the drive unit; and vice versa.

In still another aspect the present disclosure relates to a kit comprising a first drug delivery device and comprising a second drug delivery device as described above. The first and the second drug delivery devices each comprise a drive unit as described above. The first drug delivery device comprises a first container unit for a first drug container. The first container unit comprises a first counter interface with an electrically encoded first counter contact element. The second drug delivery device comprises a second container unit for a second drug container. The second container unit comprises a first counter interface with an electrically encoded second counter contact element.

The first counter contact element of the first container unit distinguishes from the second counter contact element. Distinguishing features of the first container unit and the second container unit are detectable by the electronic unit of the respective drive unit when the container unit is connected to the drive unit. Here, an electrical contact between the first and the second electrical contact elements of the first interface of the drive unit is modified by the counter contact element of the first counter interface.

With other examples the first and the second electrical contact elements of the first interface of the drive unit will each get into contact with a counter contact element of the first counter interface. Since the first counter contact element electrically distinguishes from the second counter contact element the electronic unit of the drive unit is capable and/or configured to detect a respective type of one of the first and second drug containers when the respective container unit is connected to the drive unit.

With some examples the various container units are electrically encoded, e.g. by the configuration of at least one electrically encoded counter contact element configured to get in electrical contact with at least one of the first electrical contact element and the second electrical contact element of the drive unit.

With other examples the container unit is mechanically encoded so as to induce various positions or movements of an electrical contact or electrical connector of the drive unit by way of which the second path distal end and the first path distal end can be electrically connected.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

### BRIEF DESCRIPTION OF THE DRAWINGS

In the following, numerous examples of a method of authenticating a user to use a drug delivery device as well as numerous hardware configurations including an authentication system, an electronic circuit, a drug delivery device, a supplementary device and numerous implementations of mobile electronic devices will be described in greater detail by making reference to the drawings, in which:

FIG. 1 schematically illustrates a longitudinal cross-section through an example of a drug delivery device,

FIG. 2 shows an enlarged view of the drive unit of the drug delivery device of FIG. 1,

FIG. 3 is an enlarged view of a portion of first and second device components,

FIG. 4 is an enlarged view of an electromechanical actuator in an unlocked or released configuration,

FIG. 5 shows the electromechanical actuator when actuated,

FIG. 6 is a diagram of a voltage in at least one of the electrical conductor path and/or in the electrical control path during a typical scenario of use,

FIG. 7 shows a further example of the drug delivery device with a protective cap provided with an energy reservoir,

FIG. 8 is an enlarged view of the distal end of the protective cap according to FIG. 7,

FIG. 9 shows an example of an interface and a counter into interface for fastening the protective cap to the drive unit or to the drug delivery device,

FIG. 10 shows an example of an interface with electrical contact elements and counter contact elements,

FIG. 11 shows another example of the interface according to FIG. 10,

FIG. 12 shows a further example of the interface according to FIG. 10,

FIG. 13 shows a further example of an interface of FIG. 10,

FIG. 14 shows a further schematic example of a conducting or supply path provided across the interface and the counter interface,

FIG. 15 shows a transverse cross-section through an example of an interface connected with a counter interface,

FIG. 16 shows an example of a counter interface provided at a proximal end of a container unit configured for fastening to the first interface of the drive unit,

FIG. 17 shows the interfaces according to FIG. 16 when engaged,

FIG. 18 shows another example of an interface and a counter interface according to FIG. 16,

FIG. 19 shows the interfaces of FIG. 18 when mutually engaged,

FIG. 20 shows a cross-section A-A before or during engagement of the interfaces and

FIG. 21 shows a cross-section A-A when the interfaces are mutually engaged,

FIG. 22 is illustrative of numerous examples of counter interfaces of different container units configured for fastening with the first interface of the drive unit,

FIG. 23 shows a cross-section through the first interface of the drive unit,

FIG. 24 is a block diagram of the drug delivery device configured to communicate with an external electronic device,

FIG. 25 shows another example of a first interface of the drive unit engaged with a counter interface of the container unit,

FIG. 26 is an enlarged illustration of the interfaces before or during mutual assembly or engagement,

FIG. 27 shows the example of FIG. 26 when the interfaces are engaged or connected,

FIG. 28 shows another example of the first interface for operating with a counter interface of a container unit before or during mutual assembly and

FIG. 29 shows the interfaces according to FIG. 28 when in a final assembly configuration,

FIG. 30 shows another example of a drive unit in longitudinal cross-section,

FIG. 31 shows a cross-section B-B according to FIG. 30,

FIG. 32 shows an enlarged view of the cross-section of FIG. 31,

FIG. 33 is a longitudinal cross-section through a further example of a drug delivery device,

FIG. 34 shows a cross-section through a drive unit of the drug delivery device of FIG. 33,

FIG. 35 is an enlarged view of a portion of FIG. 34,

FIG. 36 is a cross-section D-D viewed from a proximal side,

FIG. 37 is a cross-section C-C as seen from a distal side,

FIG. 38 shows a schematic illustration of a sliding contact arrangement at or near the proximal end of the drive unit according to FIG. 34,

FIG. 39 is a cross-section E-E according to FIG. 38,

FIG. 40 is a cross-section F-F according to FIG. 38,

FIG. 41 is a flowchart of one example of using or operating the drive unit and/or the drug delivery device.

### DESCRIPTION OF EXEMPLARY EMBODIMENTS

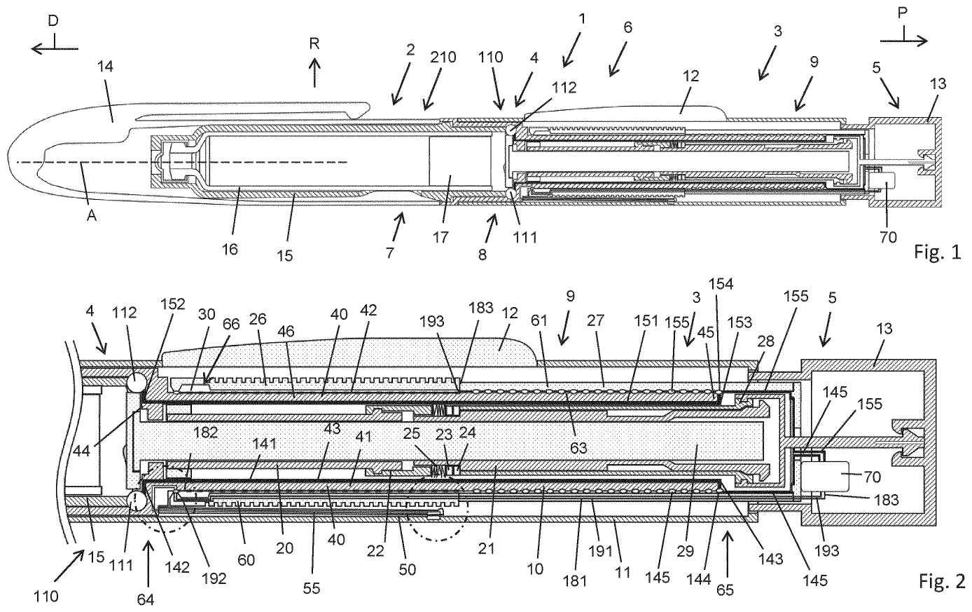

FIGS. 1, 2, 7 and 30 show exemplary embodiments of a drug delivery device 1 in a cross-sectional view. The drug delivery device 1 is a variable dose device, in which different doses of a drug to be dispensed can be set or dialed, respectively, by a user. The drug delivery device is a dial extension pen.

FIG. 1 also indicates the coordinate system used herein for specifying positions of members or elements or features. The distal direction D and proximal direction P run parallel to the longitudinal axis A. The longitudinal axis A is a main extension axis of the device 1. The radial direction R is a direction perpendicular to the longitudinal axis A and intersecting with the longitudinal axis A. A tangential direction, also referred to as circumferential direction is a direction perpendicular to the radial direction R and to the longitudinal axis A.

The drug delivery device 1 comprises a drive unit 3 with a setting mechanism and a dispense mechanism. The setting mechanism is configured for setting a drug dose and the dispense mechanism is configured for dispensing a drug dose. The functional principles of the mechanisms are explained further below.

The drive unit 3 comprises an inner body 10 and a housing element 11, in the following also referred to as outer body 11. The inner body 10 and the outer body 11 are fixedly connected to each other, i.e. they cannot be rotated or moved axially with respect to each other. The outer body 11 forms an outer surface of the drug delivery device 1 which can be touched or grabbed by a user.

The drug delivery device 1 further comprises a protective cap 14 and a user interface member 13 in form of a knob 13. The knob 13 is a dose setting member configured to be operated by a user for setting a drug dose. At the same time, the knob 13 is dose dispense member configured to be operated by a user in order to dispense a drug dose.

A drug reservoir unit 2, also referred to as a container unit 2, comprises a reservoir 16 and a reservoir holder 15 is received within the cap 14. The reservoir 16 is filled with a drug or medicament. The reservoir 16 is sealed in proximal direction P by a stopper 17.

The reservoir unit 2 is operatively coupled or connected, respectively, to the drive unit 3. The drive unit 3 is configured to enable a dispense process for dispensing a drug dose by acting on the drug reservoir 16. For dispensing a drug dose, the stopper 17 is pushed in distal direction D by a plunger rod 29 of the drive unit 3. When an injection needle 18 is connected to drug reservoir a dose of the medicament can be injected into biological tissue.

The coupling between the drive unit 3 and the reservoir unit 2 is realized by the inner body 10 being coupled to the reservoir holder 15 via a connection interface which might be a snap connection or a threaded connection. The coupling is preferably reversible. For example, the container unit 2 is axially and rotationally fixed to the inner body 10 by the coupling.

The drive unit 3 further comprises a number sleeve 26 and a dial sleeve 27 which are fixedly coupled to each other (e.g. they cannot rotate or move axially relative to each other). The dial sleeve 27 may comprise an inner thread which is engaged with an outer thread of the inner body 10. On an outer surface of the number sleeve 26, numbers may be shown. The user can see the numbers through a window 12. The window 12 may comprise a lens. The window 12 is formed in the outer body 11. The numbers visible in the window 12 indicate to a user the set/dialed dose. Due to the threaded coupling between the dial sleeve 27 and the inner body 10, the dial sleeve 27 and the number sleeve 26 are moved on a helical path in proximal direction relative to the body 10, 11 during setting a drug dose and dispensing a drug dose as will be explained further below.

The drive unit 3 also comprises a drive sleeve. The drive sleeve comprises a distal drive sleeve 20, a proximal drive sleeve 21 and a drive sleeve coupler 22 coupling the distal drive sleeve 20 to the proximal drive sleeve 21. For setting a drug dose and dispensing a drug dose, the distal drive sleeve 20 and the proximal drive sleeve 21 are fixedly coupled to each other via the drive sleeve coupler 22 so that these elements can neither rotated nor move axially relative to each other. The distal drive sleeve 20 may comprise an inner thread which is engaged with an outer thread of the plunger rod 29. An outer thread of the distal drive sleeve 20 may be engaged to an inner thread of a last dose nut 30, the function of which will be explained further below.

Furthermore, the drive unit 3 comprises a clutch 28, which is fixedly coupled to the knob 13 so that, during setting a drug dose and dispensing a drug dose, the clutch 28 and the knob 13 are not rotated or moved axially relative to each other. The clutch 28 is coupled to the proximal drive sleeve 20 via a splined engagement. This splined engagement may allow a certain axial movement of the clutch 28 relative to the proximal drive sleeve 21 but does not allow a relative rotation between these two elements.

A distal clicker 23, a proximal clicker 24 and a clutch spring 25 of the drive unit 3 are arranged between the clutch 28 and the drive sleeve coupler 22. The clutch spring 25 is coupled to the drive sleeve coupler 22 and to the distal clicker 23. The distal clicker 23 is configured to abut against the proximal clicker 24 in proximal direction P. The proximal clicker 24 is configured to abut against the clutch 28 in proximal direction P. Thus, the clutch spring 25 is configured to bias the distal clicker 23, the proximal clicker 24 and the clutch 28 in proximal direction P relative to the drive sleeve coupler 22.

The distal clicker 23 may be permanently splined to the proximal drive sleeve 21 so that a relative rotation between these two elements is prevented. However, a certain axial movement between the distal clicker 23 and the proximal drive sleeve 21 may be allowed. The proximal clicker 24 may be permanently splined to the inner body 10 so that a relative rotation between these two elements is prevented, whereas a certain relative axial movement may be allowed.

The distal face of the clutch 28 and the proximal face of the proximal clicker 24 may both be toothed so that these two faces may engage into each other. Furthermore, the distal face of the proximal clicker 24 and the proximal face of the distal clicker 23 may both be toothed so that these two toothed faces can engage into each other. A proximal face of the clutch 28 may be toothed, e.g. dog toothed, and may be arranged to engage a toothed, e.g. dog toothed, distal face of the dial sleeve 27.

FIG. 1 shows the drug delivery device 1 when no dose is set (0 units/0 unit position). Dose setting may be allowed in discrete units of 1, e.g. from 0 to 80 units. For setting a desired drug dose, the user has to rotate the knob 13. This is done without pressing on the knob 13 in distal direction D. As long as one does not press on the knob 13 in distal direction D, a dog toothed engagement between the clutch 28 and the dial sleeve 27 is established due to the clutch spring 25 either biasing the clutch 28 in proximal direction P or at least preventing the clutch 28 from moving in distal direction D on its own. The dog toothed engagement between the clutch 28 and the dial sleeve 27 has as a consequence that the two elements are rotationally locked to each other so that, when the knob 13 is rotated, also the dial sleeve 27 and the number sleeve 26 are rotated. Since the dial sleeve 27 is threadedly engaged with the inner body 10, rotating the knob 13 has as a consequence that the knob 13, the clutch 28, the dial sleeve 27 and the number sleeve 26 move on a helical path in proximal direction P relative to the body 10, 11. Thereby, the numbers of the number sleeve 26 visible through the window 12 increase.

Since the proximal drive sleeve 21 is splined to the clutch 28, also the proximal drive sleeve 21 and with it the distal drive sleeve 20 and the drive sleeve coupler 22 are moved on the helical path in proximal direction P relative to the inner body 10.

The plunger rod 29 comprises two outer threads with opposite hand which overlap with each other. The plunger rod 29 is threadedly engaged with the inner thread of the distal drive sleeve 20. The threads are chosen such that during the helical movement of the distal drive sleeve 20 in proximal direction P, the plunger rod 29 does not rotate and is also not moved axially.

The last dose nut 30 may be splined to the inner body 10 and, therefore, cannot rotate relative to the inner body 10. Due to the threaded engagement of the last dose nut 30 with the distal drive sleeve 20, the last dose nut 30 is forced to move in proximal direction P during setting a drug dose. When the maximum dose has been set (e.g. 80 units—independently of whether it has been set in only one drug setting process or several drug setting processes), the last dose nut 30 establishes a rotation-lock interface with the distal drive sleeve 20 so that the last dose nut 30 can no longer rotate relative to the distal drive sleeve 20. As a consequence of this, the distal drive sleeve 20 can no longer be rotated and no further drug dose can be set. The drug delivery device 1 then has to be reset to its initial state.

During setting a drug dose, the toothed faces of the distal clicker 23 and the proximal clicker 24 facing each other ratchet over each other thereby creating a click sound which indicates to a user that a drug dose is set. For this purpose, the teeth of the two faces are preferably formed as shallow triangles so that relative rotation between the clickers 23 and 24 is possible leading to a repeated slight compression and decompression of the clutch spring 25.

After the desired dose has been set, the user can now press on the knob 13 in distal direction D in order to dispense the set drug dose. Thereby, the distally directed force on the knob 13 is transferred from the knob 13 via the clutch 28 to the proximal clicker 24, from there to the distal clicker 23 and this compresses the clutch spring 25. The two clickers 23 and 24 are now pressed against each other and their toothed faces are engaged. Relative rotation between the two clickers 23, 24 is now prevented. Since the proximal clicker 24 is splined to the inner body 10 and the distal clicker 23 is splined to the proximal drive sleeve 21, the proximal drive sleeve 21 can no longer rotate relative to the inner body 10. However, since the proximal drive sleeve 21 is also splined to the clutch 28, also the clutch 28 and the knob 13 can no longer rotate relative to the inner body 10.

The distally directed force applied to the knob 13 has as a consequence that the clutch 28 together with the knob 13 slightly moves in distal direction D relative to the dial sleeve 27 so that the clutch spring 25 is compressed, as already mentioned. The dog toothed engagement between the dial sleeve 27 and the clutch 28 is thereby released so that the dial sleeve 27 is no longer rotationally locked to the clutch 28. Therefore, when the knob 13 is pressed in distal direction D, the dial sleeve 27 together with the number sleeve 26 can still rotate relative to the inner body 10. When the knob 13 is now moved in distal direction D, a stop against the dial sleeve 27 forces the dial sleeve 27 to also move in distal direction D. Due to the threaded engagement of the dial sleeve 27 with the inner body 10, the dial sleeve 27 together with the number sleeve 26 moves on a helical path in distal direction D. Thereby, the numbers of the number sleeve 26 visible in the window 12 decrease.

At the same time, the clutch 28, the clickers 23, 24 and the drive sleeve 20, 21, 22 are forced to move in distal direction D (without rotation). The threaded engagement between the plunger rod 29 and the distal drive sleeve 20 forces the plunger rod 29 to rotate. A further threaded engagement between the plunger rod 29 and an inner thread of the inner body 10 may then force the plunger rod 29 to also move distally in order to push the stopper 17 inside the cartridge 16 in distal direction D for dispensing the set drug dose. Since the distal drive sleeve 20 is not rotate during dispensing, the last dose not 30 moves together with the distal drive sleeve 20 in distal direction D without changing its position relative to the distal drive sleeve 20.

After having dispensed the set drug dose and when the knob 13 has been completely moved back into its initial position, a new drug dose may be set by again rotating the knob 13 on a helical path in proximal direction P. During this, the plunger rod 29 does not change its position. Only when dispensing a dose, the plunger rod 29 is moved in distal direction D.

As explained with respect to FIG. 1, one user interface member in form of a knob 13 is used for setting a drug dose as well as for dispensing the drug dose. However, it is also possible to use separate user interface members for setting and dispensing a drug dose.

Further examples of the drive unit 3 to be implemented with the injection device 1 can be found in WO 2014/033195 A1 or WO 2014/033197 A1 the entirety of which being incorporated herein by reference.

FIGS. 2-33 show the drug delivery device 1 of FIG. 1 but in different views than FIG. 1 and with more details. FIG. 2 only shows the proximal part of the drug delivery device 1 in order to better illustrate some of the details.

The drug delivery device 1 is provided as an all-mechanically implemented pen-type injection device. Accordingly, a force required for injecting the medicament is entirely provided by a user of the device. The drug delivery device 1 may be implemented as a disposable device. Such devices are intended to be discarded when the medicament provided in the drug container 16 has been used up. With other examples the drug delivery device is implemented as a reusable device. Here, the drive unit 3 forming or constituting the proximal portion of the drug delivery device is provided with a connection interface 8 that provides a mechanical releasable connection with the container unit 2. The drug delivery device may further comprise a further connection interface 7 by way of which the protective cap 14 is detachably fixable to at least one of the housing 6, e.g. the drive unit housing 9 and the cartridge holder 15. Generally, the housing 6 of the drug delivery device comprises a proximally located drive unit housing 9 connected or connectable with the container unit 2. The container unit 2 comprises the cartridge holder 15 serving as a distal housing component of the drug delivery device 1. The cartridge holder 15 is typically to be covered by the protective cap 14 when the drug delivery device is not in use.

As shown in greater detail in FIG. 2 the drive unit 3 is provided with an electronic unit 70. The electronic unit 70 is arranged inside a hollow space covered by the dose button 13. It is provided near or at the proximal end 5 of the drive unit 3. At or near an oppositely located distal end the drive unit 3 comprises a first interface 110. The first interface 110 is provided with an electrical contact element 111 and a second electrical contact element 112. The first and the second electrical contact elements 111, 112 may be provided inside a receptacle at the distal end 4 of the drive unit 3. The receptacle may be sized and configured to receive a complementary shaped insert section of the cartridge holder 15.

The first electrical contact element 111 is connected with a first electrical conductor path 141, which is located on or inside a first elongated device component 40. With the presently illustrated example the first elongated device component 40 is provided by the inner body 10 of the drive unit 3. Alternatively, it may be provided by any other elongated component of the drive unit 3 as described above.

The first device component 40 comprises a somewhat tubular and elongated shape. As illustrated, the first electrical conductor path 141 extends along an inside surface 43 of the sidewall 41 of the first device component 40. Likewise, there is provided a second electrical conductor path 151. Also the second electrical conductor path 151 is provided on the inside surface 43 of the sidewall 41 of the first elongated device component 40.

The first and the second electrical contact elements 111, 112 are provided at a distally facing longitudinal end face 44 of the first housing component. Here, the longitudinal ends, i.e. the distal ends 142, 152 of the first and second electrical conductor paths 141, 151 are in permanent electrical contact with the first and second electrical contact elements 111, 112, respectively. At the opposite longitudinal end the first elongated device component 40 comprises a proximally facing longitudinal end face 45.

A proximal end 143, 153 of the first and of the second electrical conductor paths 141, 151 terminates at the respective longitudinal end face 45. As illustrated in FIG. 2, the proximal ends 143, 153 extend radially outwardly at the longitudinal end face 45 of the first device component 40.

The drive unit 3 further comprises at least a second device component 60, presently implemented as the dial sleeve 27 and/or as the number sleeve 26. The second device component 60 is also of tubular shape. Alternatively and generally, the second device component may be represented by any component of the drive unit 3 as described above, which is movable relative to the first device component 40.

The first device component 40 is located inside the second device component 60. The tubular-shaped sidewall 61 of the second device component 60 encloses an outside surface 42 of the first device component 14. Moreover, the first device component 40 and the second device component 60 are threadedly engaged. Accordingly, the outside surface 42 of the first device component 40 is provided with a threaded section 46 in threaded engagement with a correspondingly shaped threaded section 66 at the inside surface 63 of the second device component 60. In this way, the second device component 60 is movable relative to the first device component 40 along a helical path.

On the inside of the sidewall 61 of the second device component 60, e.g. towards a proximal end of the second device component 60, e.g. formed by the dial sleeve 27, there is provided a first conductor path extension 145 and a second conductor path extension 155. The first and the second conductor path extensions 145, 155 are provided on the side surface 63 of the sidewall 61 of the second device component 60.

The first conductor path extension 145 and the second conductor path extension 155 comprise a helically wound structure with numerous windings. The helically wound structure of the first conductor path extension 145 and the helically wound structure of the second conductor path extension 155 may be arranged in a nested or convoluted manner on the inside surface 63 of the sidewall 61 of the second device component 60. Towards the proximal end 65 the first and second conductor path extensions 145, 155 are rather straight shaped and are further electrically connected to the electronic unit 70.

As illustrated in FIG. 2, the proximal end 143 of the first electrical conductor path 141 is in sliding contact with the first conductor path extension 145. Details of the sliding contact 144 are further illustrated with regards to FIGS. 31 and 32. Likewise, the proximal end 153 of the second electrical conductor path 153 is provided with a sliding contact 154 being in surface contact with the second conductor path extension 155.

The helical structure of the first and the second conductor path extensions 145, 155 has the same lead as the threaded engagement between the first device component 40 and the second device component 60. In this way and when for instance the second device component 60 is subject to a helical motion relative to the first device component 40 the electrical contact between the first electrical conductor path 141 and the first electrical conductor path extension 145 is maintained. The same applies for the second conductor path 151 and the second conductor path extension 155.

In FIGS. 2-5 there is further illustrated a first electrical control path 181 and a second electrical control path 191. The first and second electrical control paths 181, 191 extend from the electronic unit 70 towards an electromechanical actuator 50 provided at a longitudinal distance from the proximal end 5 of the drug delivery device 1. The first electrical control path 181 comprises a proximal end 183 permanently connected to the electronic unit 70. Likewise, the second electrical control path 191 comprises a proximal end 193 permanently connected to the electronic control unit 70. The electronic control unit 70 is arranged or mounted at a proximal end of the second device component 60. It may be located at a proximal end face of the second device component 60.

The first electrical control path 181 and the second electrical control path 191 are located on or inside the second device component 60. They extend in longitudinal direction along or in the sidewall 61 of the second device component 60. As illustrated in FIGS. 2-5 the electromechanical actuator 50 is provided on or in the inner body 10. It may be integrally formed with the first device component 40. The electromechanical actuator 50 may comprise a bending arm 55 extending in the longitudinal direction A. It comprises a free end towards or at its proximal end. The proximal end or free end of the bending arm for 55 is provided with a radially inwardly extending protrusion 53 to engage with a complementary-shaped recess 54 provided on the second device component 60.

The electromechanical actuator 50 comprises a first magnet 51, e.g. provided on or inside the outer body 11 as illustrated in FIGS. 4 and 5. The electromechanical actuator 50 further comprises a second magnet 52 in longitudinal alignment with the magnet 51. The second magnet 52 may be implemented as an electromagnet. It is electrically connected with a first control path extension 185 and a second control path extension 195. By providing a current via the first and the second control path extensions 185, 195 the second magnet 52 generates a magnetic field, e.g. interacting with the static magnetic field of the first magnet 51 thus leading to an attraction or repelling between the first and the second magnets 51, 52.

The bending arm 55 is resiliently deformable in radial direction. By suitably driving the electromagnet 52 with a driving current there can be induced a radially directed movement of the bending arm 55 and hence of the protrusion 53. In this way and by applying a current to the electromagnet 52 there can be established or abrogated a mechanical engagement between the protrusion 53 and one of the numerous recesses 54 as provided on an outside surface of the second device component 60.

In FIG. 4, an unlocking configuration is illustrated, in which the bending arm 55 and hence its protrusion 53 is out of engagement from the recess(es) 54. In FIG. 5 actuation or activation of the electromagnet 52 leads to a radially inwardly directed movement of the protrusion 53, thereby engaging the recess 54. Since the bending arm 55 is fixedly attached to the inner body 10 the second device component 60, in particular the number sleeve 26 is locked against rotation. Accordingly, and in the locked configuration as illustrated in FIG. 5 at least one of setting of a dose and injecting of a dose is locked.

With other examples the locking mechanism 56 is activated and hence the protrusion 53 is in engagement with the recess(es) 54 when the electromechanical actuator 50 is deactivated or is in an initial state. Then and per default, the interlock and hence the locking mechanism 56 is activated. Activation of the electromechanical actuator 50, e.g. applying a current to the electromagnet 52 then leads to an unlocking of the locking mechanism 56.

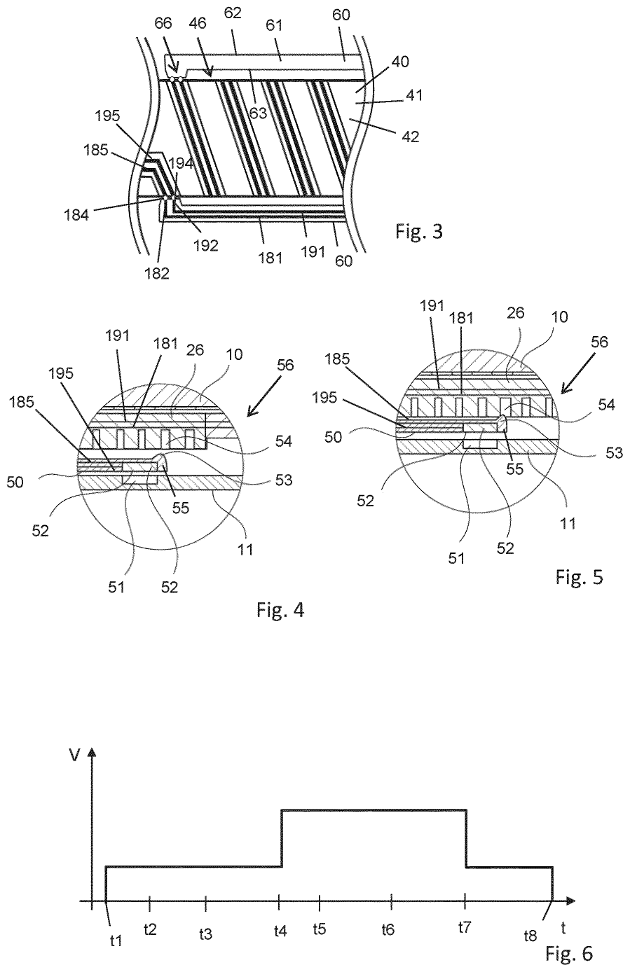

A control signal for activating or deactivating the electromagnet 52 is generated and provided by the electronic unit 70. The control signal is transmitted via the first and the second electrical control paths 181 and the mutual electrical contact configuration between the first electrical control path 181 and the first control path extension 185 as well as via the second electrical control path 191 and the second control path extension 195 as illustrated in greater detail in FIG. 3.

The first and second control path extensions 185, 195 each comprise a proximal end connected to the electromagnet 52 provided on the bending arm 55. The first and second control path extensions 185, 195 may be provided on or inside the bending arm 55. They are electrically isolated from each other. As illustrated in FIG. 2 and towards the distal end of the bending arm 55 the first and second control path extensions 185, 195 extend towards and onto the outside surface 42 of the sidewall 41 of the first elongated device component 40, which may be integrally formed with the bending arm 55.

As illustrated in FIG. 3, the first and second control path extensions 185, 195 are provided in a helical structure on the outside surface 42. The first and second control paths 181, 191 extend on or through the second device component 60, which is in threaded engagement with the first device component 40. As illustrated in FIG. 3, a distal end 182 of the first electrical control path 181 is connected to the first control path extension 185 via a sliding contact 184. Likewise, the distal end 192 of the second electrical control path 191 is connected with the second control path extension 195 of a further sliding contact 194.

The lead of the helical structure of the first and second control path extensions 185, 195 is substantially equal to the lead of the threaded section 66 of the second device component 60 which is in threaded engagement with a complementary shaped threaded section 46 of the first device component 40. In this way and when the second device component 60 is subject to a helical motion relative to the first device component 40, e.g. during setting and/or dispensing of a dose, the electrical contact between the first and second electrical control paths 181, 191 and the respective first and second control path extensions 185, 195 can be maintained.

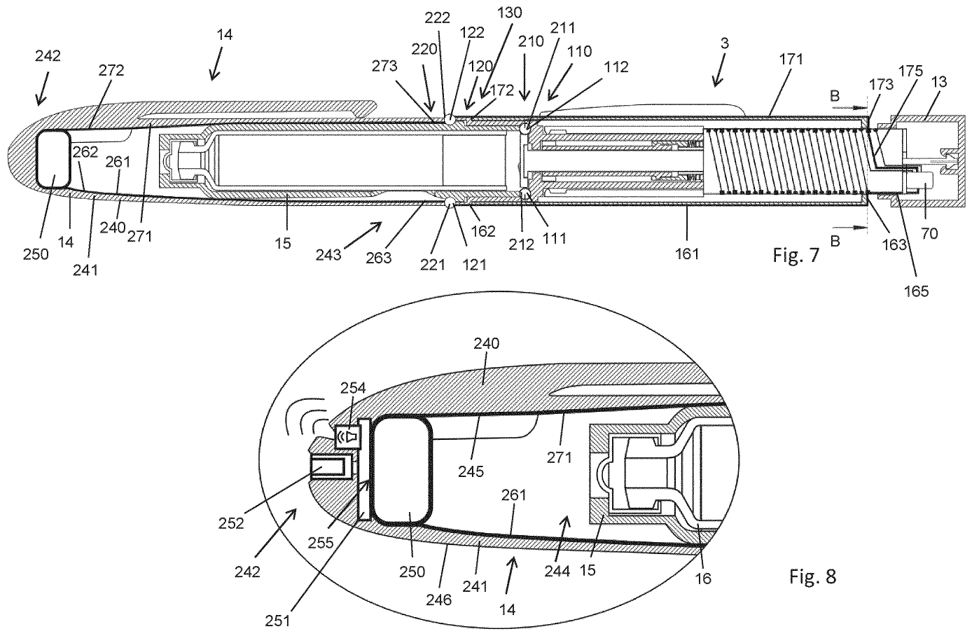

In the further example as illustrated in FIGS. 7 and 8 the drug delivery device 1 and hence the drive unit 3 is provided with a first electrical supply conductor path 161 and a second electric supply conductor path 171. The drive unit 3 comprises a second interface 120 provided at the distal end 4 of the drive unit housing 9. The second interface 120 comprises a first supply contact 121 electrically connected with the first electric supply conductor path 161. The second interface 120 further comprises a second supply contact 122 electrically connected to the second electric supply conductor path 171. Here, the second interface 120 may form or constitute an electrical interface 130.

The first and the second supply contacts 121, 122 are configured for making electrical contact with complementary shaped first and second counter supply contacts 221, 222 of a counter interface 220 provided at a proximal end of a protective cap 14. The counter supply contacts 221, 222 are electrically connected with an energy reservoir 250 provided in or on the protective cap 14. The energy reservoir 250, e.g. implemented as a secondary battery is electrically connected to the first counter supply contact 221 via a first electric supply path 261. The second counter supply contact 222 is connected with the energy reservoir 250 by a second electric supply path 271. As illustrated in FIG. 7 a distal end 262 of the first electric supply path 261 is connected with the electric energy reservoir 250. A proximal end 263 is electrically connected to the first counter supply contact 221. Likewise, a distal end 272 of the second electric supply path 271 is electrically connected to the electric energy reservoir 250.

The first and the second electric supply paths 261, 271 are provided on an inside surface 245 of the sidewall 241 of a hollow receptacle 244 of the body 240, which receptacle is sized to receive the cartridge holder 15. With other examples (not illustrated) the first and the second electric supply paths 261, 271 may be also provided on an outside surface 246 of the sidewall 241 of the body 240.

An oppositely located proximal end 273 of the electric supply path 271 is electrically connected to the second counter supply contact 222. As illustrated in FIG. 7 the first and the second supply path 261, 271 are electrically isolated from each other. They are provided on an inside surface of a sidewall 241 of a cap body 240 of the protective cap 14. The electric energy reservoir 250 is located at or near the distal end 242 of the cap body 240. The counter supply contacts 221, 222 are located at or near the proximal end 243 of the protective cap 14.

When the protective cap 14 is appropriately assembled to the drug delivery device 1 the first electric supply contact 121 is electrically connected to the first counter supply contact 221. Likewise, the second supply contact 122 is electrically connected to the second counter supply contact 222. In this way and since the first and the second electric supply conductor paths 161, 171 are electrically connected to the electronic unit 70, the electronic unit 70 can be provided with electrical energy. In particular, a battery 73 of the electronic unit 70 may be charged by electric energy as provided by the electric energy reservoir 250 of the protective cap 14.

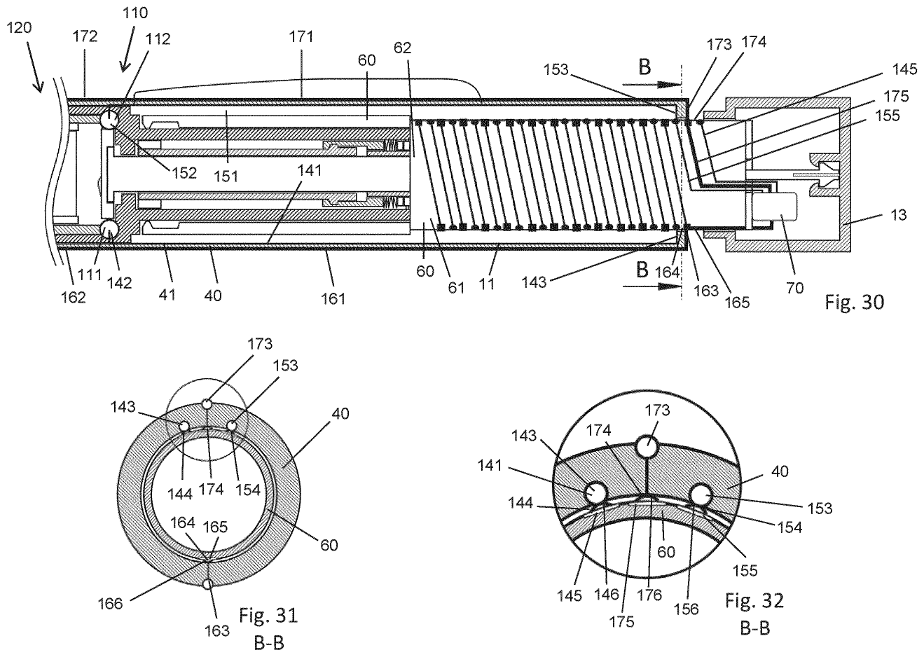

As further illustrated in FIGS. 7 and 30 the first and second electric supply conductor paths 161, 171 are indirectly connected to the electronic unit 70 via the first and second electric supply path extensions 165, 175. The first and second electric supply conductor paths 161, 171 are arranged on an outside surface of the sidewall of the outer body 11. Likewise, they may be provided on an inside surface of the outer body 11. Alternatively, they may be provided on an outside surface of the inner body 10 and hence on an outside surface of the first device component 40.

As illustrated in detail in FIG. 30 the electric supply path extensions 165, 175 are directly electrically connected to the electronic unit 70. They are provided in a helically shaped manner on the outside surface 62 of the second housing component 60. The proximal ends 163, 173 of the first and the second electric supply path 161, 171 terminate at the proximal end of the first device component 40, which is now implemented as the outer body 11.

The proximal end 163 of the first electric supply conductor path 161 is electrically connected with the first electric supply conductor path extension 165 via a sliding contact 164. Likewise, the proximal end 173 of the second electric supply conductor path 171 is electrically connected to the second electric supply conductor path extension 175 by another sliding contact 174. This way there may be provided a permanent electrical contact between the first and second electric supply contact 121, 122 provided at or near a distal end 4 of the drive unit 3 and the electronic unit 70 as provided at or near the proximal end 5 of the drive unit 3 even when the second device component 60 should be subject to a helical motion relative to the first device component 40.

As further illustrated in FIG. 8 the protective cap 14 is provided with a printed circuit board (PCB) 251 and a logic circuit 255. The PCB 251 is further provided with a power connector 252. The power connector 252 provides electrical connection to an external power supply so as to recharge the energy reservoir 250. Typically, the energy reservoir 250 is implemented as a rechargeable battery. The energy reservoir 250 typically comprises an electrical storage capacity that is substantially larger than a respective electrical storage capacity of a battery 73 of the electronic unit 70.

Moreover, there may be provided a signal generator 254 on or in the protective cap and connected to the logic circuit 255. The signal generator 254 is operable to generate and/or to transmit at least one of a visual, audible or a haptic signal. This way, the logic circuit 255 may be configured to autonomously generate an alert signal, e.g. in situations where an abrogated electrical contact with the electronic unit 70 exceeds a predefined time interval. In situations, where the protective cap 14 is not reassembled to the drive unit 3 or to the drug delivery device 1 after use the alert as generated by the signal generator 254 may remind a user of the device to put the protective cap 14 back onto the drug delivery device 1.

Moreover, the electronic circuit 255 may be operable to monitor and/or to detect the charging level of the energy reservoir 250. If the logic circuit 255 should detect that the charging level is below a predefined minimum threshold the logic circuit 255 may be configured to generate a respective alert via the signal generator 254. This way, a user of the device may be prompted to connect the protective cap 14 and hence the entire drug delivery device 1 to an external electric power supply in order to recharge the energy reservoir 250.

With the example of FIG. 30 the first and second electrical conductor paths 141, 151 are located and arranged at an inside of the outer body 11. First and second electric conductor path extensions 145, 155 are provided and arranged on the outside surface 62 of the sidewall 61 of the second housing component 60. Here, the first and second electrical conductor path extensions 145, 155 as well as the first and second electric supply conductor extensions 165, 175 are arranged in a nested or convoluted, hence interleaved manner on the outside surface 62 of the sidewall 61 of the second housing component 60.

Again, a lead of the helical structure of the various conductor path extensions 145, 155, 165, 175 is substantially identical to the lead of the threaded engagement between the first device component 40 and the second device component 60.

In the cross-section according to FIGS. 31 and 32 details of one implementation of a sliding contact 144, 154, 164, 174 are illustrated. Each sliding contact 144, 154, 164, 174 comprises at least one mechanically biased contact conductor 146, 156, 166, 176. With the example of the sliding contact 144 the contact conductor 146 is connected to one of the first electrical conductor path 141 and the first electrical conductor path extension 145. The mechanically biased contact conductor 146 may be implemented as a pliable or elastically deformable conducting tongue that is intrinsically biased in radial direction so as to maintain an electrical contact between the first electrical conductor path 141 as provided on the inside of the first device component 40 and the first electrical conductor path extension 145 as provided on the outside surface of the second device component 60.

With other examples the mechanically biased contact conductor 146 may comprise a conducting pin movable against or under the action of a biasing spring, i.e. a contact spring.

The further contact conductors 156, 166, 176 may be implemented in a likewise manner.

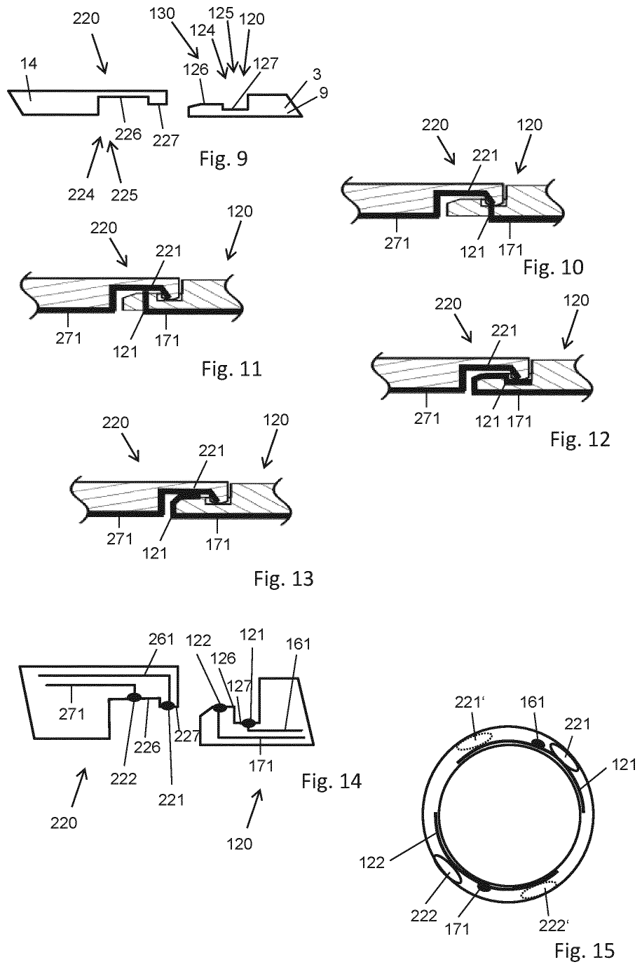

In FIGS. 9-15 there is shown a mechanical interaction between the second interface 120 with the counter interface 220 of the protective cap 14. Here, the mechanical engagement between the interfaces 120, 220 is implemented as snap features or as a snap-type connection. The second interface 120 comprises a fastener 124 implemented as a snap element 125. The snap element 125 comprises a radial protrusion 126 at its distal end longitudinally followed by a recess 127. Accordingly, the counter interface 220 comprises a complementary-shaped counter fastener 224 or a respective counter fastening structure. The counter fastener 224 comprises a complementary-shaped counter snap element 225. The counter snap element 225 comprises a protrusion 227, e.g. a radial protrusion at its proximal end followed by a recess 226. When appropriately assembled the protrusion 227 engages the recess 127 and the protrusion 126 engages the recess 226.

As further illustrated in FIGS. 10-13 the electric supply contact 121 may be provided on an inside of one of the recess 127 and the protrusion 126. Accordingly, the complementary shaped electric counter supply contact 221 is provided on one of the protrusion 227 and the recess 226. With the examples of FIGS. 10 and 12 the electric supply contact 121 terminates in the recess 127. The complementary shaped electric counter supply contact 221 terminates on a respective surface of the protrusion 227.

With the example of FIGS. 11 and 13 the electric supply contact 121 terminates on the protrusion 126. The complementary shaped counter supply contact 221 is provided across the recess 226 and may optionally even extend towards and into the protrusion 227.

In the further illustration of FIG. 14 the respective interfaces 120, 220 are provided with two electric supply contacts 121, 122 and with respective first and second counter supply contact 221, 222. Here, the first electric supply contact 121 is located in the recess 127. The second electric supply contact 122 is provided at a predefined longitudinal distance from the first supply contact 121. It is hence provided on or in the protrusion 126. Accordingly, the first counter supply contact 221 is provided on the protrusion 227 and the second counter supply contact 222 is provided on or in the recess 226. When appropriately connected and upon establishing a snap fit connection between the second interface 120 with the counter interface 220 the first electric supply contact 121 exclusively connected with the first counter supply contact 221. Likewise, the second electric supply contact 122 is in electrical contact with the second counter supply contact 222.

In the cross-section according to FIG. 15 it is illustrated that the first and second electric supply contacts 121, 122 comprise a somewhat circular or at least semicircular shape and extend along the circumference of the drive unit housing 9 or cartridge holder 15. The complementary shaped counter supply contacts 221, 222 may vary with regard to their circumferential position as indicated by the dashed lines of the first and second counter supply contacts 221′, 222′. This allows for a rotation of the protective cap 14 relative to the inner or outer body 10, 11 within a predefined extend, e.g. as specified by fastener 124 and the mutually corresponding counter fastener 224 thereby maintaining an electrical contact.

As illustrated, the circumferential extent of the first and second electric counter supply contacts 221, 222 is slightly smaller than a circumferential gap between the first and the second electric supply contacts 121, 122. This way, a short circuit between the first and the second electric supply contact 121, 122 by any of the first or second counter supply contact 221, 222 can be effectively prevented.

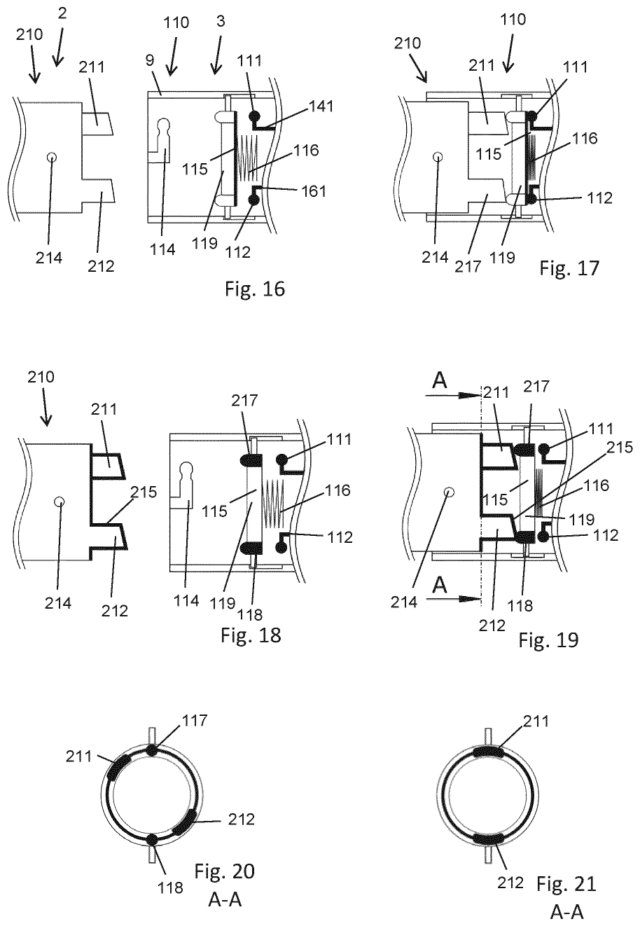

In the illustration of FIGS. 16-21 an example of a mechanical connection between the drive unit 3 and the container unit 2 is exemplary illustrated. The drive unit 3 comprises the first interface 110 complementary shaped and configured for engagement with a respective counter interface 210 as provided at the proximal end of the container unit 2. As illustrated, the first interface 110 comprises a receptacle configured and shaped to receive an insert section as provided at the proximal end of the container unit 2. The distal end of the drive unit 3, in particular a distal end of the drive unit housing 9 comprises a fastener 114 configured for engagement with a complementary shaped counter fastener 214 of the counter interface 210. As illustrated in FIGS. 16-19 the mutual connection of the fastener 114 and the counter fastener 214 may comprise or form a bayonet joint. Other types of connections, such as snap fit connections or screw-type connections are equally available or conceivable.

As further illustrated in FIG. 16 the counter interface 210 of the container unit 2 is provided with a first counter contact element 211 and with a second counter contact element 212. The first and the second counter contact elements 211, 212 comprise a somewhat beveled or slanted proximal end face. The first and second contact 111, 112 as provided on or in the first interface 110 may be selectively electrically connected by an electrical connector 115 movably disposed in or on the first interface 110.

There may be provided a carrier 119 longitudinally displaceable against or under the action of a contact spring 116. In the illustration of FIG. 16 the electrical connector 115 is provided on the carrier 119. It is out of contact from the first and the second electrical contact elements 111, 112. As the container unit 2 is connected to the drive unit 3 the counter contact elements 211, 212 will engage with the carrier 119 and urge the carrier 119 towards the proximal direction against the action of the contact spring 116. When reaching a final assembly configuration, in which the counter fastener 214 fully engages the fastener 114 the carrier 119 and the contact conductor 115 as provided on the carrier 119 have moved into a contact position with the first electrical contact element 111 and the second electric contact element 112. In this way an electrical contact between the first electric contact element 111 and the second electric contact element 112 is closed. There will be hence provided a closed conductor loop between the first electrical conductor path 141 and the second electrical conductor path 161.

With the further example of FIGS. 18 and 19 the electrical connector 115 comprises a first electrical contact element 117 and a second electrical contact element 118 located or arranged on the movable carrier 119. Here, the first and second contact elements 117, 118 of the electrical connector 115 are electrically isolated from each other. They may individually electrically contact with the first and the second electrical contacts 111, 112 of the first interface 110.

This way a mutual electrical contact between the first and the second electrical contacts 111, 112 is provided by a mechanical engagement with the first and the second counter contact elements 211, 212 that are provided with a separate contact conductor 215. Here, a surface of the first and second counter contact element 211, 212 may be provided with an electrically conducting structure or material. Moreover, the first and the second counter contact elements 211, 212 are electrically connected via the contact conductor 215. In a final assembly configuration, which is not yet reached in FIG. 19 the first electrical contact element 111 is electrically connected to the further contact element 117. The further contact element 117 is electrically connected to the counter contact element 211. The counter contact element 211 is permanently electrically connected to the counter contact element 212. Also, the counter contact element 212 is in electrical contact with the further contact element 118 of the electrical connector 115, which in turn is electrically connected to the second electric contact element 112.

In FIG. 20, a cross-section A-A is illustrated that represents the configuration before reaching a final assembly position and before the counter fastener 214 reaches an end position within the groove of the fastener 114. Here, the counter contact elements 211, 212 are located at a circumferential offset from the contact elements 117, 118. When reaching a final assembly position a respective contact configuration is obtained.

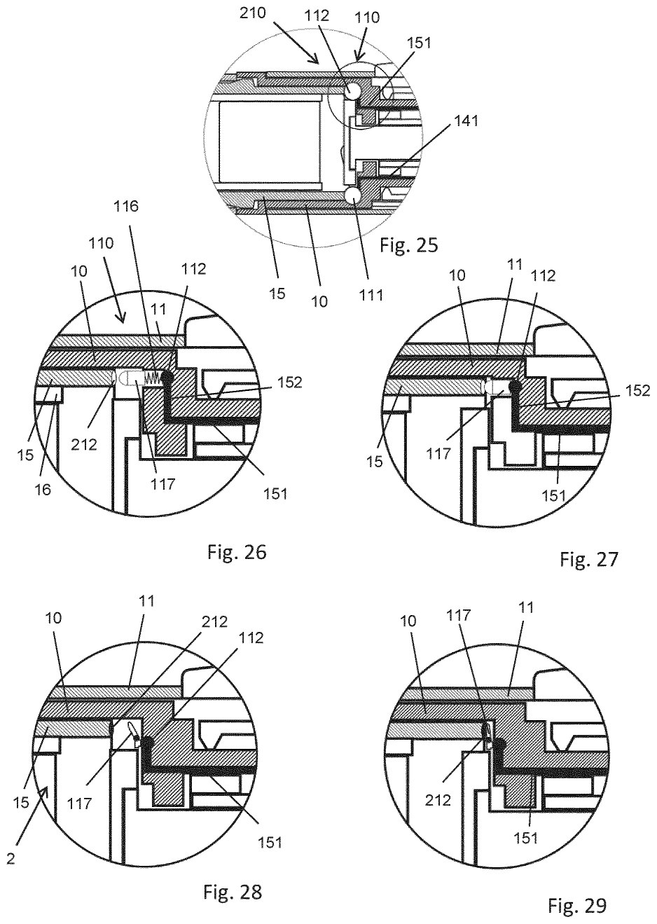

With the examples of FIGS. 25-29 another way of establishing an electrical contact between the first interface 110 and the counter interface 120 is schematically illustrated. Here, the first or second contacts 111, 112 are provided with an electrically conductive contact element 117, which is movably disposed or movably connected to the distal end 152 of the second electrical conductor path 151. The proximal end of the cartridge holder 15 may be provided with a respective counter contact element 212. The counter contact element 212 may be provided as an electrically conductive structure at a proximal end face of the sidewall of the cartridge holder 15. It may comprise an annular closed conductive ring structure on the proximal end or end face of the cartridge holder 15. In the configuration as illustrated in FIG. 26 the final assembly position has not yet been reached. Here, the counter contact element 212 and the contact element 117 are out of engagement.

When arriving in the final assembly configuration as shown in FIG. 27 the contact element 117 is in surface contact with the counter contact element 212. Moreover, the contact element 117 is mechanically biased by the contact spring 116. The contact spring 116 may be made of an electrically conductive material and may provide a respective transmission of electrical signals between the contact element 117 and the electrical conductor path 151.

With the further example as illustrated in FIGS. 28 and 29 the contact element 117 of the second electrical contact element 112 is implemented as a pliable or elastically deformable tongue, which is deformable or pivotable towards the proximal direction as it engages with the counter contact element 212 provided on the proximal end of the container unit 2. The counter contact element 212 may comprise an annular structure at the proximal end of the sidewall of the cartridge holder 15. In the proximal contact position it is electrically connected to both contact elements 111, 112. This way, the presence of a cartridge holder 15 and the correct mounting of the cartridge holder 15 to the drive unit 2 can be electronically detected.

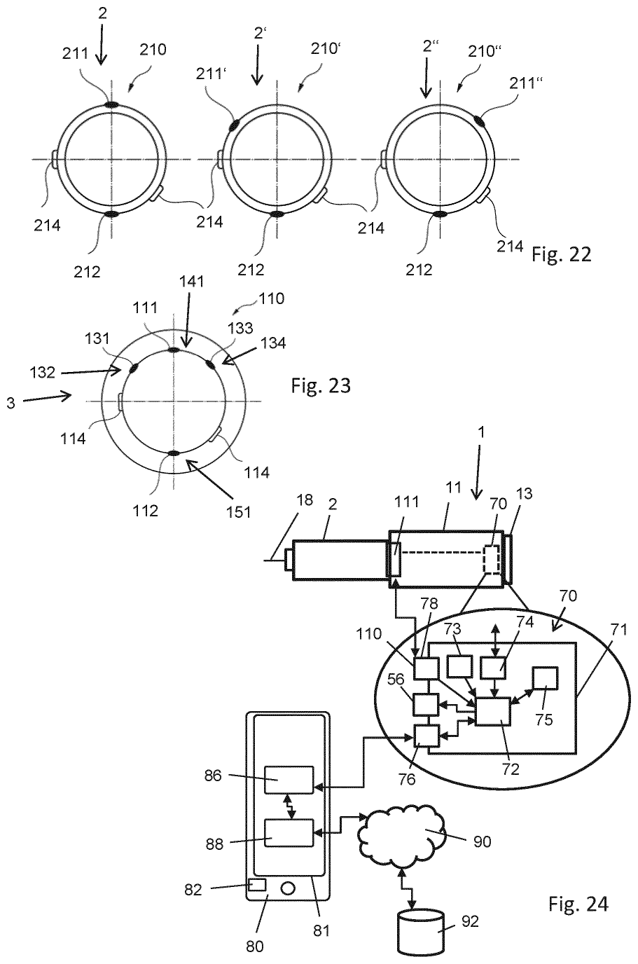

FIG. 22 is illustrative of a series of differently configured counter interfaces 210, 210′, 210″ of a series of different types of container units 2, 2′, 2″ which are typically equipped with a different drug container 16 and/or with a different medicament. The counter interfaces 210, 210′, 201″ are electrically encoded by the position or orientation of the counter contact elements 211, 212 as illustrated in FIG. 22. Moreover, the counter interfaces 210, 210′, 210″ are provided with an identical counter fastener 214 configured for mechanical engagement with a complementary shaped fastener 114 as the first interface 110 of the drive unit 3. In the present illustration there are provided two fastener 114 at a particular angular offset.

The counter fasteners 214 of the various container units 2, 2′, 2″ are provided at a respective angular offset. This way, the counter interfaces 210, 210′, 210″ can be only fastened in one discrete orientation with regard to the longitudinal axis as an axis A of rotation.

As illustrated in FIG. 23 the first interface 110 comprises not only a first electrical contact element 111 and a second electrical contact element 112 but also a third electrical contact element 131 and a fourth electrical contact element 133. The third contact element 131 is connected with a third longitudinal extending electrical conductor path 132. The fourth contact element 133 is connected with a respective for electrical conductor path 134. These supplemental conductor paths 133, 134 may be connected to the electronic unit 70 in the same or like a conducting manner as the first and second electrical conductor paths 141, 151.

With the numerous container units and counter interfaces 210, 210′, 210″ as shown in FIG. 22 there is always provided a common counter contact element 212 that is located in the same position relative to the counter fastener 214. With the numerous counter interfaces 210, 210′, 210″ the position of the further counter contact element 211, 211′, 211″ varies with regard to the circumferential direction relative to the counter contact element 212 and/or relative to the counter fasteners 214.

Only one of the contact elements 131, 111, 133 may be electrically connected to the counter contact element 211, 211′, 211″. For instance, with the counter interface 210, the counter contact element 211 is electrically connected with the counter contact element 212. With the counter interface 210′, the counter contact element 212 is electrically connected with the counter contact element 211′ and with the counter interface 210″ the counter contact element 212 is electrically connected with the counter contact element 211″.

Now and when the counter interface 210 is connected to the first interface 110 the electric contact element 112 gets in electrical contact with the counter contact element 212. Moreover, the contact element 111 gets in electrical contact with the counter contact element 211. Accordingly, the electronic unit 70 may sense or detect an electrical contact between the first and the second electric contact element 111, 112 and may thus identify the presence and/or correct assembly of the container unit 2 and the drive unit 3.

When the further counter interface 210′ is engaged with the first interface 110 the counter contact element 211′ gets in electrical contact with the third electric contact element 131. The counter contact element 212 gets in electrical contact with the second contact element 112. Accordingly, the electronic unit 70 may detect an electrical connection between the second and the third contact element 112, 131 and may thus identify the respective counter interface 210′.

Furthermore, and when the container unit 2 is equipped with the counter interface 210″ is assembled or connected with the first interface 110 there is provided an electrical contact between the second contact element 112 and the second counter contact element 212. In addition, there will be provided electrical contact between the counter contact element 211″ and the fourth contact element 133. Accordingly, and when the counter contact element 212 is electrically connected with the counter contact element 211″ there will be provided an electrical contact between the second contact element 112 and the fourth contact element 133. This electrical contact may be detected and sensed by the electronic circuit 70, which is accordingly able to distinguish between the differently electrically encoded container unit 2, 2′, 2″.

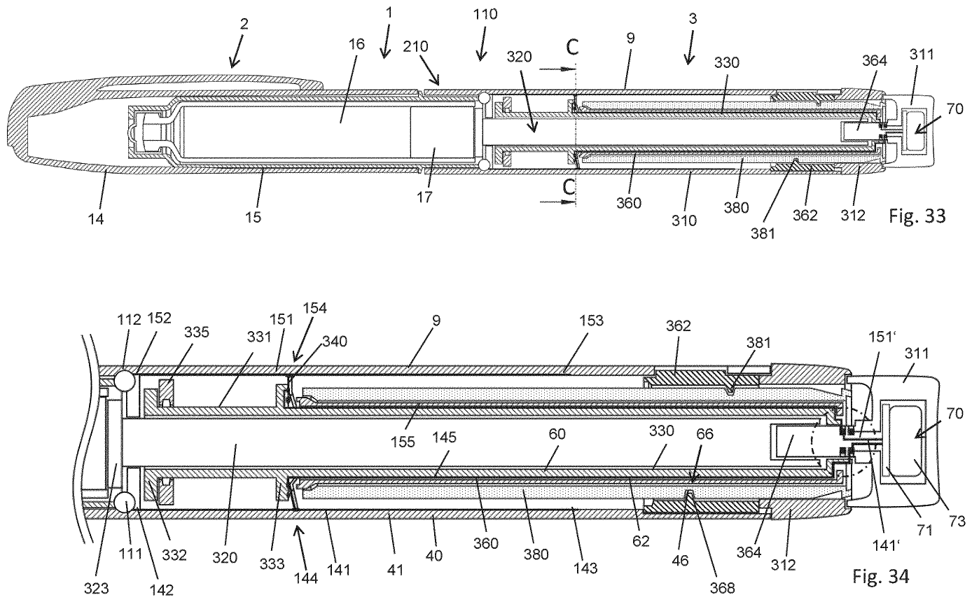

In FIGS. 33-40 there is illustrated another example of an injection device 1 provided with an electronic unit 70 at its proximal end and provided with an electrically encoded container unit 2. The device 1 of FIGS. 33-40 comprises a different type of drive unit 3 compared to the examples if FIGS. 1-32. This particular drive unit is explained in more detail e.g. in WO2004/078239A1, WO 2004/078240A1 or WO 2004/078241A1 the entirety of which being incorporated herein by reference. Here, reference numerals used before in connection with the drive unit 3 as described above in FIGS. 1-32 have the same or a similar function.

The drive unit 3 comprises a number sleeve 380 that is configured to move when a dose dial 312 is turned, in order to provide a visual indication of a currently set dose. The dose dial 312 is rotated on a helical path with respect to the housing 9 when turned during setting and/or dispensing or expelling of a dose.

The injection device 1 may be configured so that turning the dosage knob 312 causes a mechanical click sound to provide acoustical feedback to a user. The number sleeve 80 mechanically interacts with the stopper 17 in the cartridge or drug container 16. During delivery of dose, the dose dial 312 is turned to its initial position in an axial movement, that is to say without rotation, while the number sleeve 380 is rotated to return to its initial position, e.g. to display a dose of zero units.

The injection device 1 of FIG. 33 may be implemented as a disposable injection device.

A flange like support of the housing 9 comprises a threaded axial through opening threadedly engaged with a first thread or distal thread of a piston rod 320. The distal end of the piston rod 320 comprises a bearing on which a pressure foot is free to rotate with the longitudinal axis of the piston rod 320 as an axis of rotation. The pressure foot is configured to axially abut against a proximally facing thrust receiving face of the stopper 17 of the container 16. During a dispensing action the piston rod 320 rotates relative to the housing 9 thereby experiencing a distally directed advancing motion relative to the housing.

The piston rod 320 is further provided with a second thread at its proximal end. The distal thread and the proximal thread are oppositely handed. There is further provided a drive sleeve 330 having a hollow interior to receive the piston rod 320. The drive sleeve 330 comprises an inner thread threadedly engaged with the proximal thread of the piston rod 320. Moreover, the drive sleeve 330 comprises an outer threaded section 331 at its distal end. The threaded section 331 is axially confined between a distal flange portion 332 and another flange portion 333 located at a predefined axial distance from the distal flange portion 332. Between the two flange portions 332, 333 there is provided a last dose limiter 335 in form of a semi-circular nut having an internal thread mating the threaded section 331 of the drive sleeve 330.

The last dose limiter 335 further comprises a radial recess or protrusion at its outer circumference to engage with a complementary-shaped recess or protrusion at an inside of the sidewall of the housing 9. In this way the last dose limiter 335 is splined to the housing 9, e.g. to first device component 40 according to the present disclosure. A rotation of the drive sleeve 330 in a dose incrementing direction or clockwise direction during consecutive dose setting procedures leads to an accumulative axial displacement of the last dose limiter 335 relative to the drive sleeve 330.

There is further provided an annular spring 340 that is in axial abutment with a proximally facing surface of the flange portion 333. Moreover, there is provided a tubular-shaped clutch 360. At a first end the clutch 360 is provided with a series of circumferentially directed saw teeth. Towards a second opposite end of the clutch 360 there is located a radially inwardly directed flange.

Furthermore, there is provided a dose dial sleeve also denoted as number sleeve 380. The number sleeve 380 is provided outside of the spring 340 and the clutch 360 and is located radially inward of the housing 9. A helical groove 381 is provided about an outer surface of the number sleeve 380. The housing 9 is provided with a dosage window through which a part of the outer surface of the number sleeve 380 can be seen. The housing 9 is further provided with a helical rib 368 at an inside sidewall portion of an insert piece 362, which helical rib 368 is to be seated in the helical groove 381 of the number sleeve 380. The tubular shaped insert piece 362 is inserted into the proximal end of the housing 9. It is rotationally and axially fixed to the housing 9. There are provided first and second stops on the housing 9 to limit a dose setting procedure during which the number sleeve 380 is rotated in a helical motion relative to the housing 9.

Thus, the helical rib 368 may represent a threaded portion 46 of the first device component 40. The respective groove 381 is a complementary shaped threaded portion 66 of the second device component 60.

The dose dial 312 in form of a dose dial grip is disposed about an outer surface of the proximal end of the number sleeve 380. An outer diameter of the dose dial 312 typically corresponds to and matches with the outer diameter of the housing 9. The dose dial 312 is secured to the number sleeve 380 to prevent relative movement there between. The dose dial 312 is provided with a central opening.

The trigger 311, also denoted as dose button is substantially T-shaped. It is provided at a proximal end of the injection device 1. A stem 364 of the trigger 311 extends through the opening in the dose dial 312, through an inner diameter of extensions of the drive sleeve 330 and into a receiving recess at the proximal end of the piston rod 320. The stem 364 is retained for limited axial movement in the drive sleeve 330 and against rotation with respect thereto. A head of the trigger 11 is generally circular. A trigger side wall or skirt extends from a periphery of the head and is further adapted to be seated in a proximally accessible annular recess of the dose dial 312.

To dial a dose a user rotates the dose dial 312. With the spring 340 and the clutch 360 engaged, the drive sleeve 330, the spring 340, the clutch 360 and the number sleeve 380 rotate with the dose dial 312. Torque is transmitted through saw teeth between the spring 340 and the clutch 360. The helical groove 381 on the number sleeve 380 and a helical groove in the drive sleeve 330 have the same lead. This allows the number sleeve 380 to extend from the housing 9 and the drive sleeve 330 to climb the piston rod 320 at the same rate. At a limit of travel a radial stop on the number sleeve 380 engages either with a first stop or a second stop provided on the housing 9 to prevent further movement in a first sense of rotation, e.g. in a dose incrementing direction. Rotation of the piston rod 320 is prevented due to the opposing directions of the overall and driven threads on the piston rod 20.

The last dose limiter 335 keyed to the housing 9 is advanced along the threaded section 331 by the rotation of the drive sleeve 330.

During dialing or setting of a dose a ratchet mechanism allows and supports a rotation of the number sleeve 380 relative to the clutch 360 along a second sense of rotation, which rotation is accompanied by a regular clicking of a flexible arm of the clutch 360. An angular momentum applied to the number sleeve 380 along the first sense of rotation is unalterably transferred to the clutch 360. Here, the mutually corresponding ratchet features of the ratchet mechanism provide a torque transmission from the number sleeve 380 to the clutch 360.

When the desired dose has been dialed the user may simply dispense the set dose by depressing the trigger 311. This displaces the clutch 360 axially with respect to the number sleeve 380 causing dog teeth thereof to disengage. However, the clutch 360 remains keyed in rotation to the drive sleeve 330. The number sleeve 380 and the dose dial 312 are now free to rotate in accordance with the helical groove 381.

The axial movement deforms the flexible arm of the spring 340 to ensure the saw teeth cannot be overhauled during dispense. This prevents the drive sleeve 330 from rotating with respect to the housing 9 though it is still free to move axially with respect thereto. The deformation is subsequently used to urge the spring 340 and the clutch 360 back along the drive sleeve 330 to restore the connection between the clutch 360 and the number sleeve 380 when the distally directed dispensing pressure is removed from the trigger 311.

The longitudinal axial movement of the drive sleeve 330 causes the piston rod 320 to rotate through the through opening of the support of the housing 9, thereby to advance the stopper 17 in the container 16. Once the dialed dose has been dispensed, the number sleeve 380 is prevented from further rotation by contact of at least one stop extending from the dose dial 312 with at least one corresponding stop of the housing 9. A zero dose position may be determined by the abutment of one of axially extending edges or stops of the number sleeve 380 with at least one or several corresponding stops of the housing 9.

The drive unit housing 9 comprises an elongated tubular shaped sleeve. It comprises the first interface 110 provided with the first electrical contact element 111 and the second electrical contact element 112 as described above. There is further provided a first electrical conductor path 141 provided on an inside of the sidewall 41 of the first device component 40. The drive sleeve 30 as described above represents a second device component 60 as described herein, which is movably disposed inside the first device component 40. On an outside surface 62 of the second device component 60, hence on an outside surface of the tubular shaped drive sleeve 330 there are provided first and second electrical conductor path extensions 145 and 155.

The first and second electrical conductor path extensions 145, 155 are permanently electrically connected to the first and second electrical conductor paths 141, 151 via respective sliding contacts 144, 154. Here, the sliding contact 144, 154 is provided by the spring 340. The spring 340 as illustrated in FIG. 37 comprises a somewhat disc-shaped structure. It is in longitudinal sliding engagement with the first device component 40. Here, the spring 340 comprises first and second radially outwardly extending protrusions 341, 342 slidingly disposed in complementary shaped recesses 343, 345 on an inside of the sidewall 41. This way the spring 340 is allowed to slide along the longitudinal axis A but is prevented against a rotation relative to the first device component 40.

In a bottom of the first radial recess 343 there is provided the first electrical conductor path 141. In the bottom of the further recess 345 there is provided the second electrical conductor path 151. This way, the spring 340 is electrically connected to both, the first electrical conductor path 141 and the second electrical conductor path 151.

On the disc-shaped spring 340 there are provided contact conductors 146′, 156′. The contact conductors 146′, 156′ extend with one end into one of the protrusions 341, 342. There, they extend towards a radial outer edge of the respective protrusions 341, 342. On the outer radial edge of the protrusions 341, 342 there is provided the sliding contact 144, 154 with the first and second electrical conductor paths 141, 151. In the opposite direction the contact conductors 146′, 156′ extend on the planar-shaped conductor disc 148 formed by the planar-shaped spring 340.

The first contact conductor 146′ comprises a semicircular or arc-shaped structure with a first diameter. The second contact conductor 156′ also extends in a semicircular geometry on the conductor disc 148. The first contact conductor 146′ is provided on a first conductor disc portion 148′. The second contact conductor 156's provided on a second conductor disc portion 148″. The first and second conductor disc portions 148′, 148″ are electrically isolated from each other by an isolator 149 effectively separating the conductor disc 148. Alternatively, and when the conductor disc 148 should be made of a non-conducting material such a separation by way of an isolator 149 may not be necessary.

As illustrated the isolator 149 divides the first protrusion 341 and the second protrusion in two parts, wherein the first part belongs to the first conductor disc portion 148′ and wherein the second part belongs to the second conductor disc portion 148″. As further illustrated in FIG. 37 the radial extent and/or the diameter of the first contact conductor 146′ and the radial extent or diameter of the second contact conductor 156 differ from each other. In the present illustration the diameter of the semicircular second contact conductor 156′ is larger than a diameter of the first contact conductor 146′.

In FIG. 36 there is shown the proximal abutment surface of the proximal flange 333 of the drive sleeve 330 and hence of the second device component 60 as seen from the proximal direction. The flange 333 is provided with a circular and hence annular shaped further contact conductor 146″ and another circular shaped further contact conductor 156″. The diameter or shape of the further contact conductor 146″ matches the diameter and radial position of the first contact conductor 146′ provided on the conductor disc 148. The diameter and radial position of the annular shaped contact conductor 156″ matches in size and shape with the second contact conductor 156′.

When the spring 340 and hence the conductor disc 148 is in axial abutment with the flange 333 the first contact conduct conductor 146′ is in electrical contact with the further contact conductor 146″. Moreover, the second contact conductor 156′ is in electrical contact with the further contact conductor 156″.

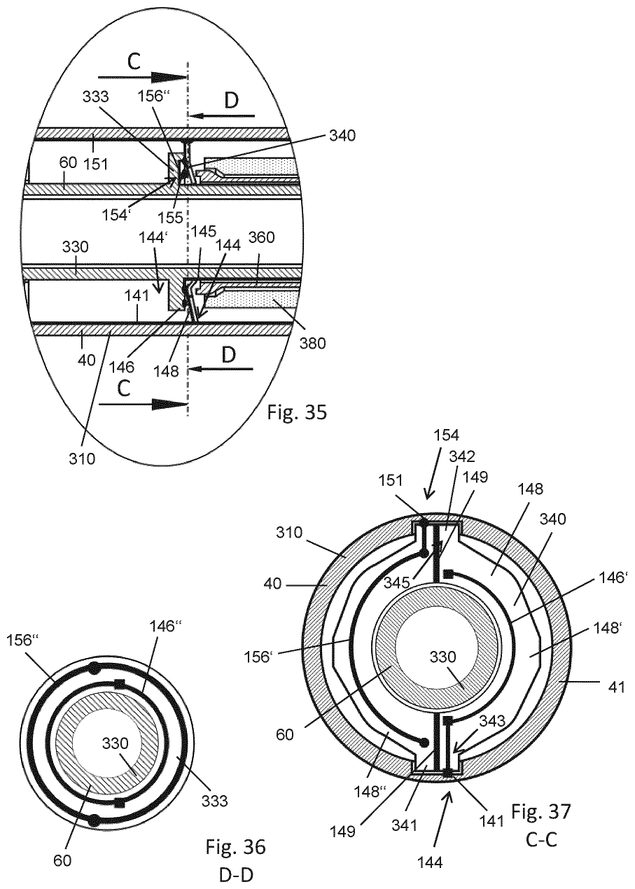

As illustrated in FIG. 35 the further contact conductor 146″ is in permanent electrical contact with the first conductor path extension 145 extending on the outside surface of the second tubular shaped device component 60. The further annular shaped contact conductor 156″ is in permanent electrical connection with the second conductor path extension 155. In this way there is provided a permanent electrical contact between the first and second electrical conductor paths 141, 151 with their respective first and second conductor path extensions 145, 155.

As illustrated in FIGS. 34 and 38-40 the first and second conductor path extensions 145, 155 extend towards the proximal end of the drive sleeve 330 and hence to the proximal end of the second device component 60. There, the proximal end of the second device component 60 comprises a receptacle to receive the stem 364 of the trigger button 311. Inside the trigger button 311 there is provided construction space for the electronic unit 70. The electronic unit 70 is mounted on a printed circuit board 71. It may be provided with a battery 73, which may be implemented as a rechargeable battery as described before in connection with the example as described with regard to FIGS. 1-32.

Inside the stem 364 there extends a first and a second electric path prolongation 141′, 151′. Towards their proximal end the conductor path extensions 145, 155 each terminate in a conductor branch or in two conductor branches 147, 157 that are in sliding engagement with an outside facing surface portion of a notch 365, 366 as provided on the outside surface of the stem 364. Each notch 365, 366 is provided with another contact conductor 146′″, 156′ extending all around the groove or notch of the stem 364. At a dedicated longitudinal position as shown in the cross section E-E of FIG. 39 the conductor path prolongation 151′ extending longitudinally through the stem 364 is electrically connected to the another contact conductor 156′ as provided on the outside of this notch 366. There, oppositely located branches 157 of the conductor path extension 155 are in sliding contact with the outside surface of the contact conductor 156′″. This way, there is formed a another sliding contact 154″.

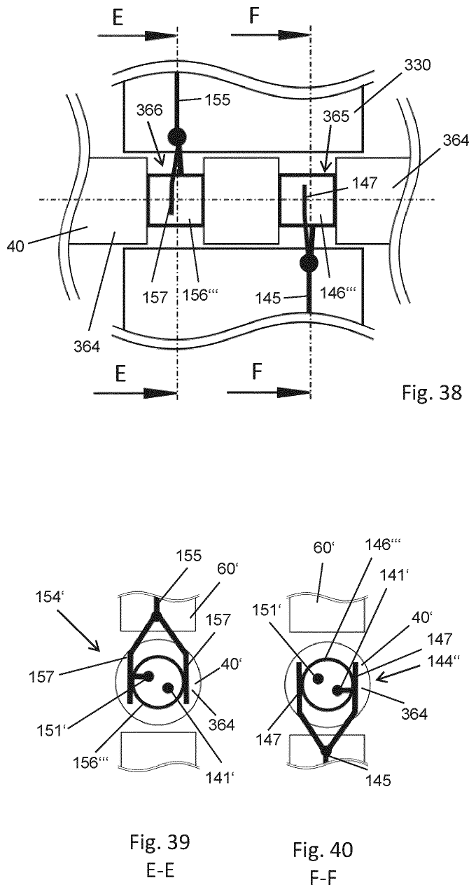

At a longitudinal offset from section E-E there is provided a section F-F as illustrated in FIGS. 38 and 40. There, it is the first conductor path prolongation 141′ that is electrically connected to the annular shaped contact conductor 146′″ as provided on the outside surface of the notch 365 or groove of the stem 364. Here, tangentially extending branches 147 of the first electric conductor path extension 145 are in sliding contact with the outside surface of the annular shaped contact conductor 146′″. In this way there is provided another sliding contact 144″ between the first electrical conductor path extension 145 and the first electric path prolongation 141′. The first electric path prolongation 141′ is directly connected to the PCB 71. Likewise, the second electric path prolongation 151′ is directly connected with the PCB 71. There is hence provided an at least twofold sliding contact.

Here, the second device component 60 is movable along a helical path during setting of a dose. Due to the semicircular shaped geometry of the arc-shaped or semicircular contact conductors 146′, 156′ there will be permanently provided an electrical contact. For dialing and/or for dispensing of a dose there may be further provided a purely rotational movement between the trigger button 311 and the drive sleeve 330, hence between the electronic unit 70 and the second device component 60. For such a purely rotational and non-longitudinal relative movement between the electronic unit 70 and the second device component 60 there are provided the further sliding contacts 144″, 154″.

Even though not explicitly shown in FIGS. 33-40 the drive unit 3 may be provided with a second interface 120 and the protective cap 14 may be also provided with an electric energy reservoir 250 as described above. In addition, the drive unit 3 may be also provided with an electromechanical actuator 50 in order to block or to lock at least one of setting of a dose and dispensing of a dose.

FIG. 24 shows a block diagram of the various injection devices 1 as described herein that are provided with an electronic unit 70. With the present example the electronic unit 70 is embedded inside the drug delivery device 1. It is arranged at or near the proximal end of the housing 6.

The electronic unit 70 comprises a printed circuit board 71 provided with a processor 72. The electronic unit 70 is further provided with a source of electric energy, typically implemented as an electric battery 73. The processor 72 and the battery 73 may be provided on opposite sides of the printed circuit board 71.

The electronic unit 70 further comprises an interface 78 electrically connected to at least one of the first and second electrical conductor paths 141, 161. With some examples, wherein the electronic unit 70 is movably disposed in the proximal housing component 14 or is connected to a dial extension movable relative to the proximal housing component the interface 78 comprises a sliding contact or is connected to a sliding contact 144, 154, 164, 174, 184, 194. This way the interface 78 and the electronic unit 70 remain in electrical contact with at least one of the interfaces 110, 120.

The electronic unit 70 may further comprise a communication interface, e.g. in form of a short range wireless communication interface 76, particularly configured to communicate with a corresponding short range communication interface 86 of an external electronic device 80.

The processor 72 is connected to the locking mechanism 56 and is operable to control operation of the locking mechanism 56.

Optionally, the electronic unit 70 is provided with a sensor 74 operable to detect or to measure movement of at least one movable component of the drive unit 3. By way of the sensor 74, which may be implemented as one of an optical sensor, a capacitive sensor, an inductive sensor, an optical sensor or as an acoustic sensor, operation of the drive unit 3 and hence operation of the injection device 1 can be supervised and monitored.

By way of the sensor 74 information regarding the size of the dose currently set, dialed or dispensed can be obtained and can be stored in a local storage 75. Data repeatedly captured or obtained during repeated and subsequent use of the injection device 10 can be stored in the storage 75. Such injection-related data stored in the storage 75 can be synchronized by the short range communication interface 76 with the external electronic device 80 and/or with an external database 92, e.g. hosted or provided by a healthcare provider.

Typically, the external electronic device 80 is implemented as a smart phone, as a smart watch or as a tablet computer. It may comprise a wide range communication interface 88 to establish a communication link to the external database 92 via a communication network 90. Typically, the external electronic device 80 is a mobile device or wearable device. It comprises a display 81 and/or a speaker 82 to communicate with a user.

The external electronic device 80 may be wirelessly paired to the electronic unit 70. Upon successful pairing a respective confirmation may be provided to a user, e.g. via the display 81 and/or via the speaker 82 of the external electronic device 80. Upon transferring of data or information between the electronic unit 70 and the external electronic device 80 a visual and/or audible confirmation may be provided to the user, e.g. by the electronic unit 70 and/or by the external electronic device 80.

The processor 72 may be configured to implement an automatic re-locking, e.g. after termination of each dispensing or in injecting procedure. Moreover, and based on signals obtainable from the sensor 74 the processor 72 may be provided with information being indicative of the remaining filling level of the medicament container 16. In this way and when the sensor 74 provides respective sensor signals that the medicament container 16 is substantially depleted or empty the processor 72 may invoke activation of the interlock 56 or may block an unlocking of the interlock 56. Removal of the medicament container 16 or insertion of the medicament container 16 as well as disconnection or reconnection of the container unit 2 may trigger an automated locking or disabling of the function of the drive unit 3 for setting and/or for dispensing of a dose.

As illustrated in FIG. 6 operation of the drug delivery devices as described herein may start at a time t1. Here, a protective cap 14 may be removed from the respective drug delivery device. Such a removal may be detected by the electronic control unit, e.g. when the electrical contact between the second interface 120 and the counter interface 220 is abrogated.

At a time t2 the electronic unit 70 may be configured to conduct a cartridge holder detection routine. Here, signals or an electrical contact provided by the first and/or second electric contact elements 111, 112 are evaluated by the electronic unit 70.

When a correct container unit 2 or cartridge holder 15 should be detected, the electronic unit 70 may switch into an activation mode. If a wrong cartridge holder 15 should be detected or if no cartridge holder should be detected the electronic unit 70 may be operable to generate an alert signal or may lock operation of the drive unit 3 by activating the interlock 56 as provided by the electromechanical actuator 50.

The electronic unit 70 in the dose button 13 may evaluate electric signal(s) from or induced by the cartridge holder 15 and may hence unlock the drive unit 3 if a correct or intended cartridge holder or container unit 2 should be present. The verification of a correct container unit is provided on the basis of the electrically detectable coding of the cartridge holder 15. In addition, there may be also conducted a user verification or an authentication procedure, e.g. by a wireless communication link with the external electronic device 80 at a time t3.

If all these requests are approved and if the locking mechanism 56 of the drive unit 3 as provided by the electromechanical actuator 50 should be locked the electronic unit 70 may generate an unlocking control signal and may transmit the respective unlocking or control signal via first and second electric control path 181, 191 thereby unlocking the drive unit 3.

As described above the control paths 181, 191 may coincide or may be electrically connected to the conductor paths 141, 151. Actuation of the electromechanical actuator 50 may then only require application of an increased voltage or current to activate or to switch the electromagnet 52. Thereafter, at a time t5 a user may set a dose of appropriate size and at a time t6 the user may conduct a respective injection procedure. At the end of an injection, hence at a time t7 the interlock of the drive unit 3 may be reactivated, e.g. by modifying the control signal from the electronic unit 70. At a time t8 re-attaching of the protective cap 14 may be detected.

In effect, the total number of conducting paths can be reduced by using only one common electric circuit for the cartridge holder detection signal and for the control signal for actuating the electromechanical actuator 50. As illustrated in FIG. 6 removing the protective cap 14 may wake up the device 1. Initially, a rather low voltage signal will be applied on the first and second electrical conductor paths 141, 151, e.g. for the purpose of cartridge holder detection and/or for the purpose of communication with the external electronic device 80. After verification of the cartridge holder 15 and/or after authentication of the user a high(er) voltage signal is applied to unlock the device.

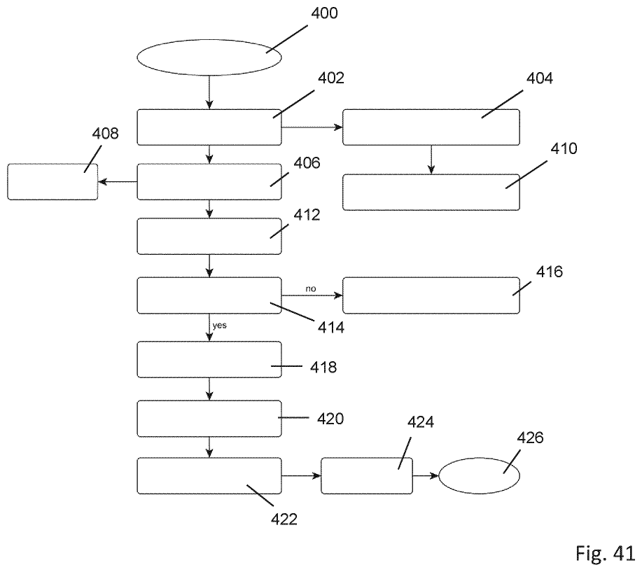

The numerous method steps of conducting an electrically implemented cartridge holder verification are further illustrated in the flowchart of FIG. 41. In step 400 the device is in a sleep mode. In step 402 the protective cap 14 is detached or removed. The injection device 1 and hence the electronic unit 70 is switched into a wake-up mode. In step 404 a timer is started to control or to monitor a reattachment of the cap. If in step 410 it should be detected that the cap 14 has not been reattached to the device over a predefined period of time the electronic unit 70 and/or the logic circuit 255 of the protective cap 14 is operable to generate a user perceivable alert signal.

In step 406 there may be conducted a battery check. If in step 408 it should be detected that the battery is low the drive unit 3 may remain in a locked state and may thus prevent setting and/or dispensing of a dose. In step 412 and when the electronic unit 70 comprises sufficient electrical energy for proper operation of the device 1 the user verification as described above is conducted, e.g. by communicating with the external electronic device and by conducting a respective authentication routine.

In step 414 it is detected if the cartridge holder 15 and/or if the container unit 2 attached to the drive unit 3 is a correct one. If it should be detected in step 414 that the container unit 2 is improper and does not match with the drive unit 3 of with a user there is provided a respective feedback in step 416. Only if in step 414 a correct cartridge holder is detected the drive unit 3 will be unlocked through respective control signals generated and transmitted by the electronic unit 70 to the electromechanical actuator 50. In step 420 the user uses the injection device. In step 422 the protective cap 14 is reattached to the drug delivery device 1 and in step 424 the timer that has been started in step 404 will be stopped the procedure terminates with step 426, in which the device 1 returns into the sleep mode.

Reference Numbers

| | |
|---|---|
| 1 | drug delivery device |
| 2 | container unit |
| 3 | drive unit |
| 4 | distal end |
| 5 | proximal end |
| 6 | housing |
| 7 | connection interface |
| 8 | connection interface |
| 9 | drive unit housing |
| 10 | inner body |
| 11 | outer body |
| 12 | window |
| 13 | dose button |
| 14 | protective cap |
| 15 | cartridge holder |
| 16 | drug container |
| 17 | stopper |
| 18 | injection needle |
| 20 | distal drive sleeve |
| 21 | proximal drive sleeve |
| 22 | drive sleeve coupler |
| 23 | distal clicker |
| 24 | proximal clicker |
| 25 | clutch spring |
| 26 | number sleeve |
| 27 | dial sleeve |
| 28 | clutch |
| 29 | plunger rod |
| 30 | last dose nut |
| 40 | device component |
| 41 | sidewall |
| 42 | outside surface |
| 43 | inside surface |
| 44 | longitudinal end face |
| 45 | longitudinal end face |
| 46 | threaded section |
| 50 | actuator |
| 51 | magnet |
| 52 | magnet |
| 53 | protrusion |
| 54 | recess |
| 55 | bending arm |
| 56 | locking mechanism |
| 60 | device component |
| 61 | sidewall |
| 62 | outside surface |
| 63 | inside surface |
| 64 | longitudinal end |
| 65 | longitudinal end |
| 66 | threaded section |
| 70 | electronic unit |
| 71 | printed circuit board |
| 72 | processor |
| 73 | battery |
| 74 | sensor |
| 75 | storage |
| 76 | communication interface |
| 78 | interface |
| 80 | external electronic device |
| 81 | display |
| 82 | speaker |
| 86 | communication interface |
| 88 | communication interface |
| 90 | network |
| 92 | database |
| 110 | interface |
| 111 | contact element |
| 112 | contact element |
| 114 | fastener |
| 115 | electrical connector |
| 116 | contact spring |
| 117 | contact element |
| 118 | contact element |
| 119 | carrier |
| 120 | interface |
| 121 | supply contact |
| 122 | supply contact |
| 124 | fastener |
| 125 | snap element |
| 126 | protrusion |
| 127 | recess |
| 130 | electrical interface |
| 131 | contact element |
| 132 | conductor path |
| 133 | contact element |
| 134 | conductor path |
| 141 | conductor path |
| 141′ | path prolongation |
| 142 | distal end |
| 143 | proximal end |
| 144 | sliding contact |
| 145 | conductor path extension |
| 146 | contact conductor |
| 147 | conductor branch |
| 148 | conductor disc |
| 148′, 148″ | conductor disc portion |
| 149 | isolator |
| 151 | conductor path |
| 151′ | path prolongation |
| 152 | distal end |
| 153 | proximal end |
| 154 | sliding contact |
| 155 | conductor path extension |
| 156 | contact conductor |
| 157 | conductor branch |
| 161 | supply path |
| 162 | distal end |
| 163 | proximal end |
| 164 | sliding contact |
| 165 | supply path extension |
| 166 | contact conductor |
| 171 | supply path |
| 172 | distal end |
| 173 | proximal end |
| 174 | sliding contact |
| 175 | supply path extension |
| 176 | contact conductor |
| 181 | control path |
| 182 | distal end |
| 183 | proximal end |
| 184 | sliding contact |
| 185 | control path extension |
| 191 | control path |
| 192 | distal end |
| 193 | proximal end |
| 194 | sliding contact |
| 195 | control path extension |
| 210 | counter interface |
| 211 | counter contact element |
| 212 | counter contact element |
| 214 | counter fastener |
| 215 | contact conductor |
| 220 | counter interface |
| 221 | counter supply contact |
| 222 | counter supply contact |
| 224 | counter fastener |
| 225 | counter snap element |
| 226 | recess |
| 227 | protrusion |
| 240 | cap body |
| 241 | sidewall |
| 242 | distal end |
| 243 | proximal end |
| 250 | energy reservoir |
| 251 | printed circuit board |
| 252 | power connector |
| 254 | signal generator |
| 255 | logic circuit |
| 261 | supply path |
| 261 | distal end |
| 262 | proximal end |
| 271 | supply path |
| 271 | distal end |
| 272 | proximal end |
| 311 | trigger |
| 312 | dose dial |
| 320 | piston rod |
| 330 | drive sleeve |
| 331 | threaded section |
| 332 | flange portion |
| 333 | flange portion |
| 335 | last dose limiter |
| 340 | spring |
| 341 | recess |
| 342 | protrusion |
| 343 | recess |
| 345 | protrusion |
| 360 | clutch |
| 362 | insert piece |
| 364 | stem |
| 365 | notch |
| 366 | notch |
| 368 | rib |
| 380 | number sleeve |
| 381 | groove |

1. A drive unit (3) of a drug delivery device (1) configured for setting and dispensing of a dose of a medicament, the drive unit (3) comprising:
a drive unit housing (9) extending along a longitudinal axis (A), the drive unit housing (9) comprising a distal end (4) and a proximal end (5),
a first interface (110) provided at the distal end (4), configured to connect with at least one of a container unit (2) and a protective cap (14), the first interface (110) comprising at least a first electrical contact element (111, 112),
an electronic unit (70) arranged on or inside the drive unit housing (9),
a first elongated device component (40) extending along the longitudinal axis (A),
a first electrical conductor path (141) located on or inside the first elongated device component (40), the first electrical conductor path (141) comprising a first path distal end (142) and a first path proximal end (143), wherein the first path proximal end (143) is connected to the electronic unit (70) and wherein the first path distal end (142) is connected to the first electrical contact element (111, 112),
wherein the electronic unit (70) is operable to execute an electronically implemented function in response to receive an electric detection signal via the first electrical conductor path (141).

2. The drive unit (3) of claim 1, further comprising a second electrical contact element (112) and a second electrical conductor path (151) electrically isolated from the first electrical conductor path (141), the second electrical conductor path (151) comprising a second path distal end (152) and a second path proximal end (153), wherein the second path proximal end (153) is connected to the electronic unit (70) and wherein the second path distal end (152) is connected to the second electrical contact element (112)

3. The drive unit (3) of claim 2, wherein the second path distal end (152) is connectable to the first path distal end (142) and via a first counter interface (210) of one of the container unit (2) and the protective cap (14) when the first counter interface (210) is engaged with the first interface (110).

4. The drive unit (3) of claim 2 or 3, wherein the second path distal end (152) is connectable to the first path distal end (142) via an electrical connector (115) movably arranged on or in the first interface (110).

5. The drive unit (3) of any one of the claims 2 to 4, wherein the second electrical conductor path (151) is located on or inside the first elongated device component (40).

6. The drive unit (3) of any one of the preceding claims, further comprising a second device component (60) movable relative to the first device component (40) for at least one of setting of the dose and dispensing of the dose of the medicament, wherein the electronic unit (70) is arranged on or in the second device component (60) and wherein a first electrical conductor path extension (145) located on or inside the second device component (60) is electrically connected to the electronic unit (70) and is electrically connected to the first electrical conductor path (141).

7. The drive unit (3) of claim 6, wherein the second device component (60) is movable relative to the first device component (40) along a first movement direction and wherein at least one of the first electrical conductor path (141) and the first electrical conductor path extension (145) extends along the first movement direction or parallel to the first movement direction.

8. The drive unit (3) of claim 6 or 7, wherein the first electrical conductor path (141) and the first electrical conductor path extension (145) are permanently electrically connected via a first sliding contact (144).

9. The drive unit (3) of claim 8, wherein the first sliding contact (144) comprises at least one mechanically biased contact conductor (146) connected to one of the first electrical conductor path (141) and the first electrical conductor path extension (145) and being in sliding surface contact with the other one of the first electrical conductor path (141) and the first electrical conductor path extension (145).

10. The drive unit (3) of any one of the preceding claims 6 to 9, wherein the first device component (40) and the second device component (60) are threadedly engaged and wherein the second device component (60) is movable relative to the first device component (40) along a helical path.

11. The drive unit (3) of any one of the preceding claims, further comprising an electromechanical actuator (50) electrically connected to the electronic unit (70) via a first electrical control path (181) and a second electrical control path (191) electrically isolated from the first electrical control path (181), wherein the electronic unit (70) is operable to selectively activate or deactivate the electromechanical actuator (50) by generating and transmitting at least one control signal to the electromechanical actuator (50) via the first and second electrical control paths (181, 191).

12. The drive unit (3) of claim 11, wherein the electronic unit (70) is operable to process the electronic detection signal received from at least one of the first electrical conductor path (141) and the second electrical conductor path (151) and is further operable to generate and to transmit an electric control signal to the electromechanical actuator (50) via at least one of the first electrical control path (181) and the second electrical control path (191) on the basis of the processing of the electric detection signal.

13. The drive unit (3) of claim 11, wherein the first electrical control path (181) is electrically connected to the first electrical conductor path (141) and wherein the second electrical control path (191) is electrically connected to the second electrical conductor path (151).

14. The drive unit (3) according to any one of the preceding claims, further comprising:
a second interface (120) at the distal end (4), wherein the second interface (120) is configured to connect with the protective cap (14) and comprises a first supply contact (121) and a second supply contact (122),
a first electric supply conductor path (161) located on or inside the first elongated device component (40), the first electric supply conductor path (161) comprising a distal end (162) and a proximal end (163), wherein the proximal end (163) is connected to the electronic unit (70) and wherein the distal end (162) is connected to the first supply contact (121), and
a second electric supply conductor path (171) located on or inside the first elongated device component (40), the second electric supply conductor path (171) comprising a distal end (172) and a proximal end (173), wherein the proximal end (173) is connected to the electronic unit (70) and wherein the distal end (172) is connected to the second supply contact (122).

15. A drug delivery device (1) for setting and dispensing of a dose of a medicament, the drug delivery device comprising:
a drive unit (3) according to any one of the preceding claims,
a container unit (2) sized to accommodate a drug container (16) and comprising a first counter interface (210) configured to connect to the first interface (110) of the drive unit (3), wherein the first counter interface (210) comprising a first counter contact element (211), the first counter contact element (211) being configured:
to electrically connect to at least one of the first electrical contact element (111) and the second contact element (112) and/or
to provide an electrical contact between the first electrical contact element (111) and the second electrical contact element (112).

16. The drug delivery device according to claim 15 further comprising the drug container (16) filled with the medical and arranged inside the container unit (2).

17. A kit comprising a first drug delivery device (1) and a second drug delivery device (1′), wherein the first and the second drug delivery devices (1, 1′) each comprise a drive unit (3) according to any one of the preceding claims 1-12,
wherein the first drug delivery device (1) comprises a first container unit (2) for a first drug container (16), the first container unit (2) comprising a first counter interface (210) with an electrically encoded first counter contact element (211),
wherein the second drug delivery device (1′) comprises a second container unit (2′) for a second drug container (16′) the second container unit (2′) comprising a first counter interface (210′, 210″) with an electrically encoded second counter contact element (211′, 211″), and
wherein the first counter contact element (211) distinguishes from the second counter contact element (211′, 211″).
